(19) United States
(12) Patent Application Publication
(10) Pub. No.: US 2009/0176762 A1
(43) Pub. Date: Jul. 9, 2009

(54) JNK Inhibitors for Treatment of Skin Diseases

(75) Inventors: Chiara Ferrandi, Moncalieri (IT); Jean-Pierre Gotteland, Beaumont (FR); Christoph H. Ladel, Nyon (CH); Paola Zaratin, Milan (IT)

Correspondence Address: SALIWANCHIK LLOYD & SALIWANCHIK;A PROFESSIONAL ASSOCIATION, PO Box 142950, GAINESVILLE, FL, 32614, US

(73) Assignee: LABORATOIRES SERONO SA, Coinsins, Vaud (CH)

(21) Appl. No.: 12/301,249

(22) PCT Filed: Jun. 1, 2007
(86) PCT No.: PCT/EP07/55429

§ 371 Date: Mar. 2, 2009

Related U.S. Application Data
(60) Provisional application No. 60/879,216, filed on Jan. 8, 2007.

(30) Foreign Application Priority Data
Jun. 2, 2006 (EP) 06114920.9

(51) Int. Cl. A61K 31/5513; A61K 31/428; A61K 31/506; A61K 31/38; A61K 31/55

(52) U.S. Cl. 514/217.03; 514/367; 514/256; 514/445; 514/218

(57) ABSTRACT

This invention relates to the use of a JNK inhibitor for treating and/or preventing skin diseases selected from psoriasis and dermatitis.

### FIELD OF THE INVENTION

The present invention is in the field of skin inflammation. In particular, the invention relates to the use of JNK inhibitors in preventing and/or treating skin diseases, in particular inflammatory skin diseases and psoriasis

### BACKGROUND OF THE INVENTION

Skin inflammation or inflammatory skin conditions/disorders are generally characterized histologically by epidermal edema and clinically by vesicles (when acute), poorly marginated redness, edema (swelling), oozing, crusting, scaling, usually pruritus, and lichenification caused by scratching or rubbing. Skin inflammation related disorders include, but are not limited to psoriasis, eczema, burning and dermatitis. Skin inflammation related disorders may be contact hypersensitivity disorders such as contact dermatitis, wherein acute or chronic inflammation is produced by substances contacting the skin and causing toxic (irritant) or allergic reactions.

### Psoriasis

Psoriasis is an inflammatory skin disorder that affects between 1 and 2% of the population. It is a common chronic, recurrent disease characterised by an increased proliferation of the epidermis, and presents as well-defined thickened erythematous patches of various sizes typically covered with a silver scale.

Psoriasis varies in severity from one or two lesions to widespread dermatosis, sometimes associated with disabling arthritis or exfoliation. The cause is unknown, but the thick scaling has traditionally been attributed to increased epidermal cell proliferation and concomitant dermal inflammation. The response of psoriasis to the immunosuppressive drug cyclosporine suggests that the primary pathogenetic factor may be immunologic. Onset is usually between ages 10 and 40, but no age is exempt. A family history of psoriasis is common. Except for the psychologic stigma of an unsightly skin disease, general health is unaffected unless psoriatic arthritis, erythrodermic psoriasis, or pustular psoriasis develops.

Psoriatic arthritis (PsA), an inflammatory arthritis and the most serious disorder associated with psoriasis, affects an estimated 25% of patients with psoriasis. PsA typically involves the distal interphalangeal joints. Current therapies for psoriasis and PsA are unsatisfactory, as none are curative; moreover, the most effective agents are associated with potentially serious side effects. In all cases, psoriasis is a chronic condition that requires long-term medication.

Erythrodermic psoriasis (exfoliative psoriatic dermatitis) may be refractory to therapy. The entire cutaneous surface is red and covered with fine scales; typical psoriatic lesions may be obscured or absent. It may lead to general debility and a need for hospitalization.

Pustular psoriasis is characterized by sterile pustules and may be generalized (von Zumbusch type) or localized to the palms and soles (Barber's psoriasis); typical psoriatic lesions may be absent.

Although the aetiology and pathology of psoriasis is not well understood, the discovery that T-cells and inflammatory cytokines are centrally involved in the development of psoriasis and is leading to the development of new biological therapeutic agents to treat these conditions.

Present treatments for psoriasis, consisting of drug therapies and phototherapies, may clear lesions and relieve uncomfortable symptoms of the disease, but remissions are usually short-lived and most patients experience at least one exacerbation or relapse per year.

Topical therapies are often the initial therapeutic choice for mild-to-moderate psoriasis. Emollients and moisturisers are the first choice because they cause few side effects; they are also often used as pre-treatments for, or in combination with, other active topical agents. Keratolytic agents act by reducing hyperkeratosis and softening psoriatic scales, aiding in their removal. As with emollients, keratolytic agents are often used to supplement other therapies.

Corticosteroids are the most common treatment for psoriasis. Numerous forms of corticosteroids are available, depending on the required potency. Systemic absorption of very potent corticosteroids can cause skin atrophy, and abrupt withdrawal can cause the disease to flare. The relapse rate has been reported to vary from 35-80%, depending on the formulation and treatment regimen used.

Topical retinoids (vitamin A analogues) work by interacting with receptors that regulate gene expression within the cell, i.e., the fundamental mechanism of cell growth and differentiation. They are applied only once daily and approximately 50-60% of patients will achieve a good response after 12 weeks of treatment. The efficacy of topical retinoids can be enhanced with concomitant high-potency corticosteroids.

Systemic therapies are frequently the therapeutic choice for moderate-to-severe psoriasis. They include phototherapy, retinoids, methotrexate and immunosuppressive drugs such as cyclosporin.

Phototherapy with ultraviolet A or B light (PUVA or PUVB) is thought to block DNA replication, thereby decreasing skin proliferation. Phototherapy with UVA, has been shown to be effective; however, its use is associated with the development of skin cancer, and is therefore limited to carefully selected patients with extensively disabling psoriasis and to those over 50 years of age.

The use of systemic retinoids is mainly limited to the treatment of severe and less common forms of psoriasis. These drugs are anti-inflammatory agents that decrease skin proliferation. However, their use is associated with severe side effects, which include liver damage and foetal abnormalities.

Methotrexate is a folic acid analogue that inhibits DNA synthesis, thus stopping rapid cell division, such as that found in untreated psoriatic lesions. Oral methotrexate is the most effective treatment in severe disabling psoriasis, especially severe psoriatic arthritis or widespread erythrodermic or pustular psoriasis unresponsive to topical agents or PUVA. It is generally reserved for very severe and refractory psoriasis, as its use is associated with liver toxicity.

Immunosuppressive drugs such as cyclosporin function by inhibiting T-cells. Cyclosporin is generally used for severe plaque psoriasis; however, its use is limited to refractory patients because of its side effects, which include renal toxicity, paraesthesia and hirsutism.

Cyclosporine is extremely effective but it has potentially serious systemic side effects.

The humanized anti-CD11a antibody efalizumab or Raptiva® received marketing approval from the FDA in 2003 for the treatment for the treatment of psoriasis. It is an immunosuppressive agent that has the potential to increase the risk of infection and reactivate latent, chronic infections.

### Dermatitis

Dermatitis is also called eczema. It relates to a superficial skin inflammation, characterized histologically by epidermal edema and clinically by vesicles (when acute), poorly marginated redness, edema, oozing, crusting, scaling, usually pruritus, and lichenification caused by scratching or rubbing.

Often, eczema refers to vesicular dermatitis, but sometimes the term is restricted eczema to mean chronic dermatitis. Some also refer to dermatitis as spongiotic dermatitis because spongiosis (intraepidermal edema) is a histologic feature.

Contact Dermatitis is an acute or chronic inflammation, often asymmetric or oddly shaped, produced by substances contacting the skin and causing toxic (irritant) or allergic reactions. It may be caused by a primary chemical irritant or by an allergen (ie, a type IV delayed hypersensitivity reaction).

Primary irritants may damage normal skin or irritate existing dermatitis. Clinically recognizable changes may occur within minutes of exposure to strong irritants (eg, acids, alkalis, phenol) or may take up to several days' exposure to weak or marginal irritants (eg, soap, detergents, acetone, or even water). The mechanisms by which these irritants damage the skin are different for different agents. For example, detergents activate keratinocytes, causing them to release inflammatory cytokines.

Allergic contact dermatitis patients may become allergic to substances that they have sometimes used for years or to drugs used to treat skin diseases. Allergens are captured by Langerhans' cells (a minor subpopulation of epidermal cells), which present them to T cells. Cytokines released from keratinocytes and Langerhans' cells may also contribute to sensitivity induction. It takes between 6 and 10 days (in the case of strong sensitizers, eg, poison ivy) to years (for weaker sensitizers) for patients to become sensitized. On reexposure to the sensitizer, patients may develop pruritus and dermatitis within 4 to 12 h.

Photoallergic and phototoxic contact dermatitides require exposure to light after topical application of certain chemicals. The chemicals (phototoxins) produce an exaggerated response to sunlight by acting as photosensitizers (see polymorphous light eruptions under Photosensitivity in Ch. 119). Aftershave lotions, sunscreens, and topical sulfonamides are commonly responsible for photoallergic contact dermatitis. Phototoxic contact dermatitis is commonly caused by certain perfumes, coal tar, psoralens, and oils used in manufacturing. Photoallergic and phototoxic contact dermatitides must be differentiated from photosensitivity reactions to systemic drugs.

Contact dermatitis ranges from transient redness to severe swelling with bullae pruritus and vesiculation are common. Any skin surface exposed to an irritant or sensitizing substance (including airborne ones) may be involved. Typically, the dermatitis is limited to the site of contact but may later spread.

The course varies. If the causative agent is removed, erythema disappears within a few days to weeks and blisters dry up. Vesicles and bullae may rupture, ooze, and crust. As inflammation subsides, scaling and some temporary thickening of the skin occur. Continued exposure to the causative agent or complications (eg, irritation from or allergy to a topical drug, excoriation, infection) may perpetuate the dermatitis.

Unless the causative agent is identified and removed, treatment may be ineffective. An oral corticosteroid may be given (if not contraindicated). Antihistamines are ineffective in suppressing allergic contact dermatitis but may blunt the itching.

c-Jun N-Terminal Kinases (JNKs)

Mammalian cells respond to some extracellular stimuli by activating signaling cascades which are mediated by various mitogen-activated protein kinases (MAPKs). Despite the differences in their response to upstream stimuli, the MAP kinase cascades are organized in a similar fashion, consisting of MAP kinase kinase kinases (MAPKKK or MEKK), MAP kinase kinases (MAPKK or MKK) and MAP kinases (MAPK). MAP kinases are a broad family of kinases, which includes c-Jun N-Terminal kinases (JNKs), also known as “stress-activated protein kinases” (SAPKs), as well as extracellular signal regulated kinases (ERKs) and p38 MAP kinases. Each of these three MAP kinases sub-families is involved in at least three different but parallel pathways conveying the information triggered by external stimuli. The JNK signaling pathway is activated by exposure of cells to environmental stress—such as chemical toxins, radiation, hypoxia and osmotic shock—as well as by treatment of cells with growth factors or pro-inflammatory cytokines—such as tumour necrosis factor alpha (TNF-α) or interleukin-1 beta (IL-1β).

Two MAP kinase kinases (known as MKKs or MAPKKs), i.e. MKK4 (known also as JNKK1) and MKK7, activate JNK by a dual phosphorylation of specific threonine and tyrosine residues located within a Thr-Pro-Tyr motif on the activation loop on the enzyme, in response to cytokines and stress signals. Even further upstream in the signaling cascade, MKK4 is known to be activated itself also by a MAP kinase kinase kinase, MEKK1 through phosphorylation at serine and threonine residues.

Once activated, JNK binds to the N-terminal region of transcription factor targets and phosphorylates the transcriptional activation domains resulting in the up-regulation of expression of various gene products, which can lead to apoptosis, inflammatory responses or oncogenic processes (Davis, 2000).

Some transcription factors known to be JNK substrates are the Jun proteins (c-jun, JunB and Jun D), the related transcription factors ATF2 and ATFa, Ets transcription factors such as Elk-1 and Sap-1, the tumor suppressor p53 and a cell death domain protein (DENN).

Three distinct JNK enzymes have been identified as products of the genes JNK1, JNK2 and JNK3 and ten different isoforms of JNK have been identified (Gupta et al., 1996). JNK1 and -2 are ubiquitously expressed in human tissues, whereas JNK3 is selectively expressed in the brain, heart and testes (2). Each isoform binds to the substrates with different affinities, suggesting, in vivo, a substrate specific regulation of the signaling pathways by the different JNK isoforms.

Activation of the JNK pathway has been documented in a number of disease processes, thus providing a rationale for targeting this pathway for drug discovery. In addition, molecular genetic approaches have validated the pathogenic role of this pathway in several diseases.

For example, auto-immune and inflammatory diseases derive from the inappropriate activation of the immune system. Activated immune cells express many genes encoding inflammatory molecules, including cytokines, growth factors, cell surface receptors, cell adhesion molecules and degradative enzymes. Many of these genes are known to be regulated by the JNK pathway, through the activation of the transcription factors c-Jun and ATF-2.

The inhibition of JNK activation in bacterial lipopolysaccharide-stimulated macrophages, effectively modulates the production of the key pro-inflammatory cytokine, TNFα (Dumitru et al., 2000).

The inhibition of JNK activation decreases the transcription factor activation responsible of the inducible expression of matrix metalloproteinases (MMPs) (Han et al., 2001), which are known to be responsible of the promotion of cartilage and bone erosion in rheumatoid arthritis and of generalized tissue destruction in other auto-immune diseases.

The JNK cascade is also activated in T cells by antigen stimulation and CD28 receptor co-stimulation (Nishina et al., 1997) and regulates the production of the IL-2 promoter (Kempiak et al., 1999). Inappropriate activation of T lymphocytes initiates and perpetuates many auto-immune diseases, including asthma, inflammatory bowel syndrome and multiple sclerosis.

In neurons vulnerable to damage from Alzheimer's disease and in CA1 neurons of patients with acute hypoxia (de la Monte et al., 2000), JNK3 protein is highly expressed. The JNK3 gene was also found to be expressed in the damaged regions of the brains of Alzheimer's patients (Zhu et al., 2001). In addition, neurons from JNK3 KO mice were found to become resistant to kainic acid induced neuronal apoptosis compared to neurons from wild-type mice.

ERK and JNK were shown to be activated in a tissue culture of psoriatic involved epidermis (Takahashi et al., 2002). However, from these in vitro data, a medical utility of JNK inhibition in psoriasis cannot be predicted

Several small molecules have been proposed as modulators of the JNK pathway.

Aryl-oxindole derivatives of respectively the generic formula (A) (WO 00/35909; WO 00/35906; WO 00/3592) and formula (B) (WO 00/64872) have been developed for the treatment of neurodegenerative diseases, inflammation and solid tumors for formula (A) and for the treatment of a broad range of disorders including, neurodegenerative diseases, inflammatory and autoimmune diseases, cardiovascular and bone disorders for formula (B).

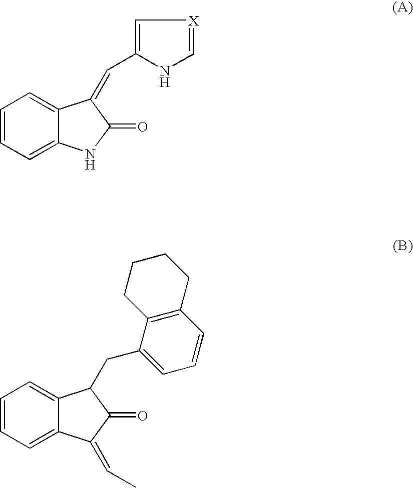

Pyrazoloanthrones derivatives of formula (C) have been reported to inhibit JNK for the treatment of neurological degenerative diseases, inflammatory and autoimmune disorders as well as cardiovascular pathologies (WO 01/12609).

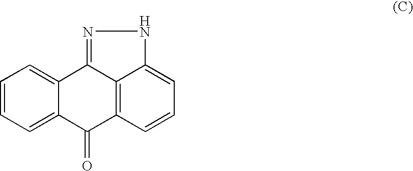

Tetrahydro-pyrimidine derivatives of formula (D) were reported to be JNK inhibitors useful in the treatment of a wide range of diseases including neurodegenerative diseases, inflammatory and autoimmune disorders, cardiac and destructive bone pathologies (WO 00/75118).

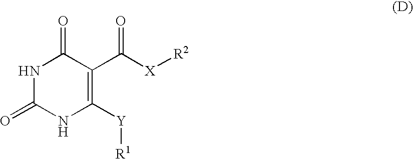

Other heterocyclic compounds of formula (E) have been proposed to inhibit protein kinases and especially c-un-N-Terminal kinases (WO 01/12621) for treating “JNK-mediated conditions” including proliferative diseases, neurodegenerative disorders, inflammatory and autoimmune disorders.

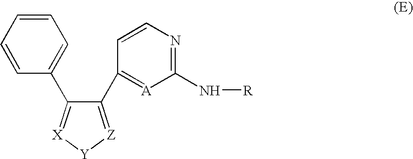

Benzazoles derivatives such as represented by formula (F) have been described as modulators of the JNK pathway for the treatment of neuronal disorders, autoimmune diseases, cancers, cardiovascular diseases (WO 01/47920, EP110957) and scleroderma (WO 03/047570) The use of similar compounds in combination with cyclosporin has been described for the treatment of a neuronal disorder, an autoimmune disease, an inflammatory disorder, cancer or a cardiovascular disease (WO 2005/097116).

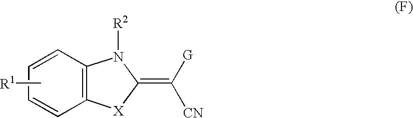

Several sulfonamide derivatives of formula (G) (WO 01/23378), sulfonyl amino acid derivatives of formula (H) (WO 01/23379) and sulfonyl hydrazide derivatives of formula (J) (WO 01/23382), were also developed to inhibit JNKs for treating neurodegenerative diseases, autoimmune disorders, cancers and cardiovascular diseases.

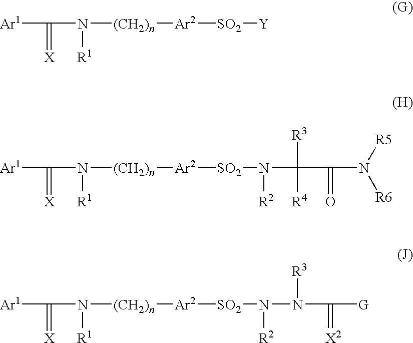

Aryl pyridine amino or aryl pyrimidine amino derivatives have been described in WO 02/079197 as modulators of the JNK pathway for the treatment of a wide range of diseases including inflammatory diseases, autoimmune diseases, destructive bone disorders, neurodegenerative diseases or a condition associated with proinflammatory cytokines.

Pyrrolotriazine derivatives have been described as kinase inhibitors, including p38 kinases and/or c-Jun N-terminal kinases for treating chronic inflammatory diseases, inflammatory bowel disease, rheumatoid arthritis, psoriasis, multiple sclerosis, endotoxin shock, osteoporosis, Alzheimer's disease, and congestive heart failure (WO 2006/047354).

Pyrazole pyridine or pyrazole pyrimidine derivatives have been described as JNK inhibitors useful for treating JNK-mediated diseases, especially neurodegenerative diseases in which all three JNK isoforms are implicated (WO02/46184).

### SUMMARY OF THE INVENTION

The present invention is based on the finding that in a murine model of skin inflammation related disorders, JNK (c-Jun Kinases) inhibitors have a beneficial effect in the treatment and/or prevention of skin diseases. The invention therefore relates to the use of a JNK inhibitor for treating and/or preventing skin diseases, preferably inflammatory skin diseases and more preferably psoriasis.

### BRIEF DESCRIPTION OF THE DRAWINGS

FIG. 1: Shows ear swelling in μm during CHS time course. ***p<0.001 vs non sensitized. One way ANOVA analysis followed by Tukey test.

FIG. 2: Showns cytokines levels (IL12p70, TNFa, IFNg, MCP-1, IL-10, IL-6, IL-5, IL-4 and IL-2) during CHS time course. *p<0.05, ***p<0.001 vs unsensitized. One way ANOVA followed by Tukey test.

FIG. 3: Intracellular signaling during CHS time course: Protein immunoblot analysis. *p<0.05 vs unsensitized. One way ANOVA followed by Tukey test.
A western blott showing phosphorilated and total c-jun, ERK, AKT, p38MAPK and caspase-3
B phosphorylated versus total c-jun and caspase-3

FIG. 4: shows the inhibition of DNFB-induced ear swelling in CHS model after JNK inhibitor p.o. administration of 10 and 30 mg/kg and dexamethasone at 1 mg/kg via sc route. °°p<0.01 vs unsensitized; *, ***p<0.05, p<0.001 vs sensitized. One way ANOVA followed by Tukey test.

### DESCRIPTION OF THE INVENTION

The following paragraphs provide definitions of the various chemical moieties that make up the compounds to be used according to the invention and are intended to apply uniformly throughout the specification and claims unless an otherwise expressly set out definition provides a broader definition.

“C₁-C₆-alkyl” refers to alkyl groups having 1 to 6 carbon atoms. This term is exemplified by groups such as methyl, ethyl, n-propyl, isopropyl, n-butyl, isobutyl, tert-butyl, n-butyl, n-pentyl, n-hexyl and the like.

“Aryl” refers to an unsaturated aromatic carbocyclic group of from 6 to 14 carbon atoms having a single ring (e.g., phenyl) or multiple condensed rings (e.g., naphthyl). Preferred aryl include phenyl, naphthyl, phenantrenyl and the like.

“C₁-C₆-alkyl aryl” refers to C₁-C₆-alkyl groups having an aryl substituent, including benzyl, phenethyl and the like.

“Heteroaryl” refers to a monocyclic heteroaromatic, or a bicyclic or a tricyclic fused-ring heteroaromatic group. Particular examples of heteroaromatic groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl or benzoquinolyl.

“C₁-_{C6}-alkyl heteroaryl” refers to C₁-C₆-alkyl groups having a heteroaryl substituent, including 2-furylmethyl, 2-thienylmethyl, 2-(1H-indol-3-yl)ethyl and the like.

“C₂-C₆-alkenyl” refers to alkenyl groups preferably having from 2 to 6 carbon atoms and having at least 1 or 2 sites of alkenyl unsaturation. Preferable alkenyl groups include ethenyl (—CH═CH₂), n-2-propenyl (allyl, —CH₂CH═CH₂) and the like.

“C₂-C6-alkenyl aryl” refers to C₂-C₆-alkenyl groups having an aryl substituent, including 2-phenylvinyl and the like.

“C₂-C₆-alkenyl heteroaryl” refers to C₂-C₆-alkenyl groups having a heteroaryl substituent, including 2-(3-pyridinyl)vinyl and the like.

“C₂-C₆-alkynyl” refers to alkynyl groups preferably having from 2 to 6 carbon atoms and having at least 1-2 sites of alkynyl unsaturation, preferred alkynyl groups include ethynyl (—C≡CH), propargyl (—CH2C≡CH), and the like.

“C₂-C₆-alkynyl aryl” refers to C₂-C₆-alkynyl groups having an aryl substituent, including phenylethynyl and the like.

“C₂-C₆-alkynyl heteroaryl” refers to C₂-C₆-alkynyl groups having a heteroaryl substituent, including 2-thienylethynyl and the like.

“C₃-C₈-cycloalkyl” refers to a saturated carbocyclic group of from 3 to 8 carbon atoms having a single ring (e.g., cyclohexyl) or multiple condensed rings (e.g., norbornyl). Preferred cycloalkyl include cyclopentyl, cyclohexyl, norbornyl and the like.

“C₁-C₆-alkyl cycloalkyl” refers to C₁-C₆-alkyl groups having a cycloalkyl substituent, including cyclohexylmethyl, cyclopentylpropyl, and the like.

“Heterocycloalkyl” refers to a C₃-C₈-cycloalkyl group according to the definition above, in which 1 to 3 carbon atoms are replaced by hetero atoms chosen from the group consisting of O, S, NR, R being defined as hydrogen or C₁-C₆ alkyl. Preferred heterocycloalkyl include pyrrolidine, piperidine, piperazine, 1-methylpiperazine, morpholine, and the like.

“C₁-C₆-alkyl heterocycloalkyl” refers to C₁-C₆-alkyl groups having a heterocycloalkyl substituent, including 2-(1-pyrrolidinyl)ethyl, 4-morpholinylmethyl, (1-methyl-4-piperidinyl)methyl and the like.

“Carboxy” refers to the group —C(O)OH.

“C₁-C₆-alkyl carboxy” refers to C₁-C₆-alkyl groups having a carboxy substituent, including 2-carboxyethyl and the like.

“Acyl” refers to the group —C(O)R where R includes H, “C₁-C₆-alkyl”, “C₂-C₆-alkenyl”, “C₂-C₆-alkynyl”, “C₃-C₈-cycloalkyl” “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl acyl” refers to C₁-C₆-alkyl groups having an acyl substituent, including 2-acetylethyl and the like.

“Acyloxy” refers to the group —OC(O)R where R includes H, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl acyloxy” refers to C₁-C₆-alkyl groups having an acyloxy substituent, including 2-(acetyloxy)ethyl and the like.

“Alkoxy” refers to the group —O—R where R includes “C₁-C₆-alkyl”, “C₂-C₆-alkenyl”, “C₂-C₆-alkynyl”, “C₃-C₈-cycloalkyl”, “Heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl alkoxy” refers to C₁-C₆-alkyl groups having an alkoxy substituent, including 2-ethoxyethyl and the like.

“Alkoxycarbonyl” refers to the group —C(O)OR where R includes “C₁-C₆-alkyl”, “C₂-C₆-alkenyl”, “C₂-C₆-alkynyl”, “C₃-C₈-cycloalkyl”, “Heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl alkoxycarbonyl” refers to C₁-C₆-alkyl groups having an alkoxycarbonyl substituent, including 2-(benzyloxycarbonyl)ethyl and the like.

“Aminocarbonyl” refers to the group —C(O)NRR′ where each R, R′ includes independently hydrogen, “C₁-C₆-alkyl”, “C₂-C₆-alkenyl”, “C₂-C₆-alkynyl”, “C₃-C₈-cycloalkyl”, “Heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl aminocarbonyl” refers to C₁-C₆-alkyl groups having an aminocarbonyl substituent, including 2-(dimethylaminocarbonyl)ethyl and the like.

“Acylamino” refers to the group —NRC(O)R′ where each R, R′ is independently hydrogen, “C₁-C₆-alkyl”, “C₂-C₆-alkenyl”, “C₂-C₆-alkynyl”, “C₃-C₈-cycloalkyl”, “Heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl acylamino” refers to C₁-C₆-alkyl groups having an acylamino substituent, including 2-(propionylamino)ethyl and the like.

“Ureido” refers to the group —NRC(O)NR′R″ where each R, R′, R″ is independently hydrogen, “C₁-C₆-alkyl”, “C₂-C₆-alkenyl”, “C₂-C₆-alkynyl”, “C₃-C₈-cycloalkyl”, “Heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”, and where R′ and R″, together with the nitrogen atom to which they are attached, can optionally form a 3-8-membered heterocycloalkyl ring.

“C₁-C₆-alkyl ureido” refers to C₁-C₆-alkyl groups having an ureido substituent, including 2-(N′-methylureido)ethyl and the like.

“Amino” refers to the group —NRR′ where each R, R′ is independently hydrogen, “C₁-C₆-alkyl”, “C₂-C₆-alkenyl”, “C₂-C₆-alkynyl”, “C₃-C₈-cycloalkyl”, “Heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”, and where R and R′, together with the nitrogen atom to which they are attached, can optionally form a 3-8-membered hetero-cycloalkyl ring.

“C₁-C₆-alkyl amino” refers to C₁-C₆-alkyl groups having an amino substituent, including 2-(1-pyrrolidinyl)ethyl and the like.

“Cyclic amino” refers to piperazinyl, piperidinyl, imidazolidinyl, imidazolinyl, imidazolyl, indolinyl, isoindolinyl, pyrazolidinyl, pyrrolidinyl “Acyclic amino” refers to the group —NRR′ where each R, R′ is independently hydrogen or “C₁-C₆-alkyl” or “aryl” or “heteroaryl” or “C₁-C₆-alkyl aryl” or “C₁-C₆-alkyl heteroaryl”, or “cycloalkyl”, or “heterocycloalkyl” and also refers to the ammonium group —N⁺RR′R″ such as defined hereinafter “Ammonium” refers to a positively charged group —N⁺RR′R″, where each R, R′, R″ is independently, “C₁-C₆-alkyl”, “C₂-C₆-alkenyl”, “C₂-C₆-alkynyl”, “C₃-C₈-cycloalkyl”, “Heterocycloalkyl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”, and where R and R′, together with the nitrogen atom to which they are attached, can optionally form a 3-8-membered heterocycloalkyl ring.

“C₁-C₆-alkyl ammonium” refers to C₁-C₆-alkyl groups having an ammonium substituent, including 2-(1-pyrrolidinyl)ethyl and the like.

“Halogen” refers to fluoro, chloro, bromo and iodo atoms.

“Sulfonyloxy” refers to a group —OSO₂—R wherein R is selected from H, “C₁-C₆-alkyl”, “C₁-C₆-alkyl” substituted with halogens, e.g., an —OSO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonyloxy” refers to C₁-C₆-alkyl groups having a sulfonyloxy substituent, including 2-(methylsulfonyloxy)ethyl and the like.

“Sulfonyl” refers to group “—SO₂—R” wherein R is selected from H, “aryl”, “heteroaryl”, “C₁-C₆-alkyl”, “C₁-C₆-alkyl” substituted with halogens, e.g., an —SO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonyl” refers to C₁-C₆-alkyl groups having a sulfonyl substituent, including 2-(methylsulfonyl)ethyl and the like.

“Sulfinyl” refers to a group “—S(O)—R” wherein R is selected from H, “C₁-C₆-alkyl”, “C₁-C₆-alkyl” substituted with halogens, e.g., an —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfinyl” refers to C₁-C₆-alkyl groups having a sulfinyl substituent, including 2-(methylsulfinyl)ethyl and the like.

“Sulfanyl” refers to groups —S—R where R includes H, “C₁-C₆-alkyl”, “C₁-C₆-alkyl” substituted with halogens, e.g., an —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”. Preferred sulfanyl groups include methylsulfanyl, ethylsulfanyl, and the like.

“C₁-C₆-alkyl sulfanyl” refers to C₁-C₆-alkyl groups having a sulfanyl substituent, including 2-(ethylsulfanyl)ethyl and the like.

“Sulfonylamino” refers to a group —NRSO₂—R′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonylamino” refers to C₁-C₆-alkyl groups having a sulfonylamino substituent, including 2-(ethylsulfonylamino)ethyl and the like.

“Aminosulfonyl” refers to a group —SO₂—NRR′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl aminosulfonyl” refers to C₁-C₆-alkyl groups having an aminosulfonyl substituent, including 2-(cyclohexylaminosulfonyl)ethyl and the like.

“Substituted or unsubstituted”: Unless otherwise constrained by the definition of the individual substituent, the above set out groups, like “alkyl”, “alkenyl”, “alkynyl”, “aryl” and “heteroaryl” etc. groups can optionally be substituted with from 1 to 5 substituents selected from the group consisting of “C₁-C₆-alkyl”, “C₂-C₆-alkenyl”, “C₂-C₆-alkynyl”, “cycloalkyl”, “heterocycloalkyl”, “C₁-C₆-alkyl aryl”, “C₁-C₆-alkyl heteroaryl”, “C₁-C₆-alkyl cycloalkyl”, “C₁-C₆-alkyl heterocycloalkyl”, “amino”, “ammonium”, “acyl”, “acyloxy”, “acylamino”, “aminocarbonyl”, “alkoxycarbonyl”, “ureido”, “aryl”, “heteroaryl”, “sulfinyl”, “sulfonyl”, “alkoxy”, “sulfanyl”, “halogen”, “carboxy”, trihalomethyl, cyano, hydroxy, mercapto, nitro, and the like. Alternatively said substitution could also comprise situations where neighbouring substituents have undergone ring closure, notably when vicinal functional substituents are involved, thus forming, e.g., lactams, lactons, cyclic anhydrides, but also acetals, thioacetals, animals formed by ring closure for instance in an effort to obtain a protective group.

“Pharmaceutically acceptable salts or complexes” refers to salts or complexes that retain the desired biological activity of the below-identified compounds of formula I and exhibit minor or no undesired toxicological effects. Examples of such salts include, but are not restricted to acid addition salts formed with inorganic acids (e.g. hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and poly-galacturonic acid. Said compounds can also be administered as pharmaceutically acceptable quaternary salts known by a person skilled in the art, which specifically include the quarter-nary ammonium salt of the formula —NR, R′, R″⁺ Z⁻, wherein R, R′, R″ is independently hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, fumarate, citrate, tartrate, ascorbate, cinnamoate, mandeloate, and diphenylacetate).

“Pharmaceutically active derivative” refers to any compound that upon administration to the recipient, is capable of providing directly or indirectly, the activity disclosed herein.

The “tautomers” of the compounds according to formula I are only those wherein R2 and/or R0 are hydrogen and which display the formulae (Ia) and (Ib).

“Enantiomeric excess” (ee) refers to the products that are obtained by an asymmetric synthesis, i.e. a synthesis involving non-racemic starting materials and/or reagents or a synthesis comprising at least one enantioselective step, whereby a surplus of one enantiomer in the order of at least about 52% ee is yielded.

“JNK” means a protein or an isoform thereof expressed by a JNK 1, JNK 2, or JNK 3 gene (Gupta et al. 1996).

“JNK-inhibitor” refers to a compound, a peptide or a protein that inhibits c-jun amino terminal kinase (JNK) phosphorylation of a JNK targeted transcription factor. The JNK-inhibitor is an agent capable of inhibiting the activity of JNK in vitro or in vivo. Such inhibitory activity can be determined by an assay or an animal model well-known in the art.

In the frame of the present invention it has been found that the administration of a JNK inhibitor has a beneficial effect in an animal model of psoriasis and allergic dermatitis.

The invention therefore relates to the use of a therapeutically effective amount of a JNK inhibitor for the manufacture of a medicament for treating and/or preventing a skin disease.

The terms “treating” and “preventing”, as used herein, should be understood as preventing, inhibiting, attenuating, ameliorating or reversing one or more symptoms or cause(s) of skin diseases, as well as symptoms, diseases or complications accompanying skin disease. When “treating” skin disease, the substances according to the invention are given after onset of the disease, “prevention” relates to administration of the substances before signs of disease can be noted in the patient.

In a preferred embodiment of the invention, the skin disease is an inflammatory skin disease.

Skin inflammation or inflammatory skin diseases are generally characterized histologically by epidermal edema and clinically by vesicles (when acute), poorly marginated redness, edema (swelling), oozing, crusting, scaling, usually pruritus, and lichenification caused by scratching or rubbing. Skin inflammation related disorders include, but are not limited to psoriasis, eczema, burning and dermatitis. Skin inflammation related disorders may be contact hypersensitivity disorders such as contact dermatitis, wherein acute or chronic inflammation is produced by substances contacting the skin and causing toxic (irritant) or allergic reactions.

In a further preferred embodiment of the invention, the skin disease is psoriasis.

“Psoriasis” refers to psoriasis in any classification and definition, as well as one or more of the symptoms of scleroderma. Psoriasis is an inflammatory skin disorder. It is a common chronic, recurrent disease characterised by an increased proliferation of the epidermis. Psoriasis is discussed in more details in the “Background of the invention” above.

In a further preferred embodiment, the skin disease is dermatitis in particular allergic dermatitis or allergic contact dermatitis.

“Allergic dermatitis” or allergic contact dermatitis is an itchy skin condition caused by an allergic reaction to material in contact with the skin. Dermatitis is discussed in more details in the “Background of the invention” above.

A “therapeutically effective amount” is such that when administered, the JNK inhibitor results in inhibition of the biological activity of JNK. The dosage administered, as single or multiple doses, to an individual will vary depending upon a variety of factor, including JNK inhibitor pharmacokinetic properties, the route of administration, patient conditions and characteristics (sex, age, body weight, health, size), extent of symptoms, concurrent treatments, frequency of treatment and the effect desired. Adjustment and manipulation of established dosage ranges are well within the ability of those skilled, as well as in vitro and in vivo methods of determining the inhibition of JNK in an individual.

The JNK inhibitors may be of formula (I)

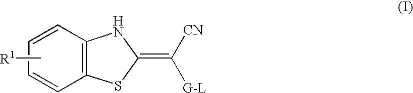

Said compounds are disclosed in WO 01/47920 (Applied Research Systems ARS Holding NV) in which benzazoles derivatives of formula (A) are described in particular for the treatment of neuronal disorders, autoimmune diseases, cancer and cardiovascular diseases:

In the compounds according to formula (I):

G is an unsubstituted or substituted pyrimidinyl group.

L is an unsubstituted or substituted C₁-C₆-alkoxy, or an amino group, or an unsubstituted or a substituted 3-8 membered heterocycloalkyl, containing at least one heteroatom selected from N, O, S (e.g. a piperazine, a piperidine, a morpholine, a pyrrolidine).

R1 is selected from the group comprising or consisting of hydrogen, sulfonyl, amino, unsubstituted or substituted C₁-C₆-alkyl, unsubstituted or substituted C₂-C₆-alkenyl, unsubstituted or substituted C₂-C₆-alkynyl or C₁-C₆-alkoxy, unsubstituted or substituted aryl (e.g. phenyl), halogen, cyano or hydroxy.

Preferably R¹ is H or C₁-C₃ alkyl (e.g. a methyl or ethyl group).

Formula (I) also comprises its tautomers, its geometrical isomers, its optically active forms as enantiomers, diastereomers and its racemate forms, as well as pharmaceutically acceptable salts thereof. Preferred pharmaceutically acceptable salts of the formula (I) are acid addition salts formed with pharmaceutically acceptable acids like hydrochloride, hydrobromide, sulfate or bisulfate, phosphate or hydrogen phosphate, acetate, benzoate, succinate, fumarate, maleate, lactate, citrate, tartrate, gluconate, methanesulfonate, benzenesulfonate, and para-toluenesulfonate salts.

More specifically, the benzothiazole acetonitriles of formula (I) comprise the tautomeric forms, e.g. the below ones:

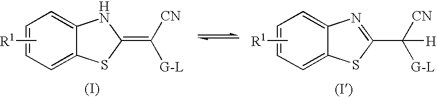

A specific embodiment of the present invention consists in benzothiazole acetonitriles of formula (Ia) in its tautomeric forms, e.g. the below ones:

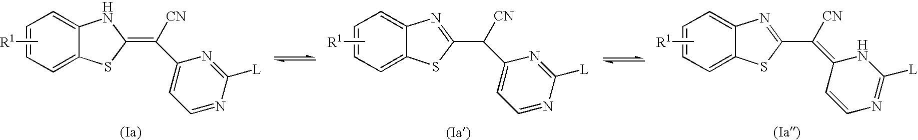

R¹ and L are as defined for formula (I).

According to a specific embodiment, the moiety L is an amino group of the formula —NR³R⁴ wherein R³ and R⁴ are each independently from each other H, unsubstituted or substituted C₁-C₆-alkyl, unsubstituted or substituted C₂-C₆-alkenyl, unsubstituted or substituted C₂-C₆-alkynyl, unsubstituted or substituted C₁-C₆-alkoxy, unsubstituted or substituted aryl, unsubstituted or substituted heteroaryl, unsubstituted or substituted saturated or unsaturated 3-8-membered cycloalkyl, unsubstituted or substituted 3-8-membered heterocycloalkyl, (wherein said cycloalkyl, heterocycloalkyl, aryl or heteroaryl groups may be fused with 1-2 further cycloalkyl, heterocycloalkyl, aryl or heteroaryl group), unsubstituted or substituted C₁-C₆-alkyl aryl, unsubstituted or substituted C₁-C₆-alkyl heteroaryl, unsubstituted or substituted C₂-C₆-alkenyl aryl, unsubstituted or substituted C₂-C₆-alkenyl heteroaryl, unsubstituted or substituted C₂-C₆-alkynyl aryl, unsubstituted or substituted C₂-C₆-alkynyl heteroaryl, unsubstituted or substituted C₁-C₆-alkyl cycloalkyl, unsubstituted or substituted C₁-C₆-alkyl heterocycloalkyl, unsubstituted or substituted C₂-C₆-alkenyl cycloalkyl, unsubstituted or substituted C₂-C₆-alkenyl heterocycloalkyl, unsubstituted or substituted C₂-C₆-alkynyl cycloalkyl, unsubstituted or substituted C₂-C₆-alkynyl heterocycloalkyl.

Alternatively, R³ and R⁴ may form a ring together with the nitrogen to which they are attached.

In a specific embodiment, R³ is hydrogen or a methyl or ethyl or propyl group and R⁴ is selected from the group consisting of unsubstituted or substituted (C₁-C₆)-alkyl, unsubstituted or substituted C₁-C₆ alkyl-aryl, unsubstituted or substituted C₁-C₆-alkyl-heteroaryl, unsubstituted or substituted cycloalkyl, unsubstituted or substituted heterocycloalkyl, unsubstituted or substituted aryl or heteroaryl and unsubstituted or substituted 4-8 membered saturated or unsaturated cycloalkyl.

In a further specific embodiment, R³ and R⁴ form a substituted or unsubstituted piperazine or a piperidine or a morpholine or a pyrrolidine ring together with the nitrogen to which they are bound, whereby said optional substituent is selected from the group consisting of unsubstituted or substituted C₁-C₆-alkyl, unsubstituted or substituted C₂-C₆-alkenyl, unsubstituted or substituted C₂-C₆-alkynyl, unsubstituted or substituted C₁-C₆-alkoxy, unsubstituted or substituted aryl, unsubstituted or substituted heteroaryl, unsubstituted or substituted saturated or unsaturated 3-8-membered cycloalkyl, unsubstituted or substituted 3-8-membered heterocycloalkyl, (wherein said cycloalkyl, heterocycloalkyl, aryl or heteroaryl groups may be fused with 1-2 further cycloalkyl, heterocycloalkyl, aryl or heteroaryl group), unsubstituted or substituted C₁-C₆-alkyl aryl, unsubstituted or substituted C₁-C₆-alkyl heteroaryl, unsubstituted or substituted C₂-C₆-alkenyl aryl, unsubstituted or substituted C₂-C₆-alkenyl heteroaryl, unsubstituted or substituted C₂-C₆-alkynyl aryl, unsubstituted or substituted C₂-C₆-alkynyl heteroaryl, unsubstituted or substituted C₁-C₆-alkyl cycloalkyl, unsubstituted or substituted C₁-C₆-alkyl heterocycloalkyl, unsubstituted or substituted C₂-C₆-alkenyl cycloalkyl, unsubstituted or substituted C₂-C₆-alkenyl heterocycloalkyl, unsubstituted or substituted C₂-C₆-alkynyl cycloalkyl, unsubstituted or substituted C₂-C₆-alkynyl heterocycloalkyl.

In a specific embodiment L is selected from:

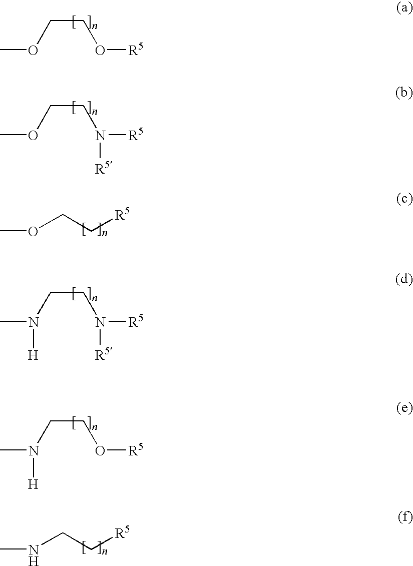

wherein n is 1 to 3, preferably 1 or 2.

R⁵ and R^{5′} are independently selected from each other from the group consisting of H, substituted or unsubstituted C₁-C₆ alkyl, substituted or unsubstituted aryl or substituted or unsubstituted heteroaryl, substituted or unsubstituted C₁-C₆ alkyl-aryl and substituted or unsubstituted C₁-C₆-alkyl-heteroaryl.

Compounds wherein L is moiety (d) are particularly preferred.

Specific examples of compounds of formula (I) include the following:
1,3-benzothiazol-2-yl(2,6-dimethoxy-4-pyrimidinyl)acetonitrile
1,3-benzothiazol-2-yl(2-{[2-(1H-imidazol-5-yl)ethyl]amino}-4-pyrimidinyl)acetonitrile
1,3-benzothiazol-2-yl[2-(1-piperazinyl)-4-pyrimidinyl]acetonitrile
1,3-benzothiazol-2-yl[2-(4-benzyl-1-piperidinyl)-4-pyrimidinyl]acetonitrile
1,3-benzothiazol-2-yl[2-(4-methyl-1-piperazinyl)-4-pyrimidinyl]acetonitrile
1,3-benzothiazol-2-yl[2-(4-morpholinyl)-4-pyrimidinyl]acetonitrile
1,3-benzothiazol-2-yl[2-(methylamino)-4-pyrimidinyl]acetonitrile
1,3-benzothiazol-2-yl(2-{4-[2-(4-morpholinyl)ethyl]-1-piperazinyl}-4-pyrimidinyl)-acetonitrile
1,3-benzothiazol-2-yl{2-[4-(benzyloxy)-1-piperidinyl]-4-pyrimidinyl}acetonitrile
1,3-benzothiazol-2-yl[2-(4-hydroxy-1-piperidinyl)-4-pyrimidinyl]acetonitrile
1,3-benzothiazol-2-yl(2-{[2-(dimethylamino)ethyl]amino}-4-pyrimidinyl)acetonitrile
1,3-benzothiazol-2-yl[2-(dimethylamino)-4-pyrimidinyl]acetonitrile
1,3-benzothiazol-2-yl{2-[(2-methoxyethyl)amino]-4-pyrimidinyl}acetonitrile
1,3-benzothiazol-2-yl{2-[(2-hydroxyethyl)amino]-4-pyrimidinyl}acetonitrile
1,3-benzothiazol-2-yl[2-(propylamino)-4-pyrimidinyl]acetonitrile
1,3-benzothiazol-2-yl(2-{[3-(1H-imidazol-1-yl)propyl]amino}-4-pyrimidinyl)acetonitrile
1,3-benzothiazol-2-yl[2-(1-pyrrolidinyl)-4-pyrimidinyl]acetonitrile
1,3-benzothiazol-2-yl{2-[(2-phenylethyl)amino]-4-pyrimidinyl}acetonitrile
1,3-benzothiazol-2-yl(2-{[2-(2-pyridinyl)ethyl]amino}-4-pyrimidinyl)acetonitrile
1,3-benzothiazol-2-yl{2-[(2-pyridinylmethyl)amino]-4-pyrimidinyl}acetonitrile
1,3-benzothiazol-2-yl{2-[4-(1H-1,2,3-benzotriazol-1-yl)-1-piperidinyl]-4-pyrimidinyl}acetonitrile
1,3-benzothiazol-2-yl{2-[4-(2-pyrazinyl)-1-piperazinyl]-4-pyrimidinyl}acetonitrile
1,3-benzothiazol-2-yl{2-[4-(2-pyrimidinyl)-1-piperazinyl]-4-pyrimidinyl}acetonitrile
1,3-benzothiazol-2-yl(2-{[2-(3-pyridinyl)ethyl]amino}-4-pyrimidinyl)acetonitrile
1,3-benzothiazol-2-yl(5-bromo-2-{[2-(dimethylamino)ethyl]amino}-4-pyrimidinyl)-acetonitrile
1,3-benzothiazol-2-yl{2-[(2-morpholin-4-ylethyl)amino]pyrimidin-4-yl}acetonitrile
1,3-benzothiazol-2-yl[2-(4-{3-[(trifluoromethyl)sulfonyl]anilino}piperidin-1-yl)pyrimidin-4-yl]acetonitrile
1,3-benzothiazol-2-yl(2-{[3-(2-oxopyrrolidin-1-yl)propyl]amino}pyrimidin-4-yl)-acetonitrile
1,3-benzothiazol-2-yl(2-{methyl[3-(methylamino)propyl]amino}pyrimidin-4-yl)acetonitrile
1,3-benzothiazol-2-yl(2-{[3-(4-methylpiperazin-1-yl)propyl]amino}pyrimidin-4-yl)-acetonitrile
1,3-benzothiazol-2-yl{2-[(3-morpholin-4-ylpropyl)amino}pyrimidin-4-yl]acetonitrile
1,3-benzothiazol-2-yl(2-{[2-(1-methyl-1H-imidazol-4-yl)ethyl]amino}pyrimidin-4-yl)acetonitrile
1,3-benzothiazol-2-yl(2-{[2-(1H-indol-3-yl)ethyl]amino}pyrimidin-4-yl)acetonitrile
1,3-benzothiazol-2-yl(2-{[2-(4-hydroxyphenyl)ethyl]amino}pyrimidin-4-yl)acetonitrile
tert-butyl({4-[1,3-benzothiazol-2-yl(cyano)methyl]pyrimidin-2-yl}amino)acetate
{2-[(3-aminopropyl)amino]pyrimidin-4-yl}(1,3-benzothiazol-2-yl)acetonitrile
{2-[(2-aminoethyl)amino]pyrimidin-4-yl}(1,3-benzothiazol-2-yl)acetonitrile
1,3-benzothiazol-2-yl(2-{[3-(dimethylamino)propyl]amino}pyrimidin-4-yl)acetonitrile
1,3-benzothiazol-2-yl{2-[(2-piperidin-1-ylethyl)amino]pyrimidin-4-yl}acetonitrile
1,3-benzothiazol-2-yl(2-{[2-(1-methyl-1H-imidazol-5-yl)ethyl]amino}pyrimidin-4-yl)acetonitrile
1,3-benzothiazol-2-yl[2-(benzylamino)pyrimidin-4-yl]acetonitrile isopropyl 3-({4-[1,3-benzothiazol-2-yl(cyano)methyl]pyrimidin-2-yl}amino)propanoate
1,3-benzothiazol-2-yl{2-[(3-hydroxypropyl)amino]pyrimidin-4-yl}acetonitrile
1,3-benzothiazol-2-yl{2-[(pyridin-3-ylmethyl)amino]pyrimidin-4-yl}acetonitrile
1,3-benzothiazol-2-yl{2-[(pyridin-4-ylmethyl)amino]pyrimidin-4-yl}acetonitrile
tert-butyl 4-[2-({4-[1,3-benzothiazol-2-yl(cyano)methyl]pyrimidin-2-yl}amino)-ethyl]phenylcarbamate
(2-{[2-(4-aminophenyl)ethyl]amino}pyrimidin-4-yl)(1,3-benzothiazol-2-yl)acetonitrile
1,3-benzothiazol-2-yl(2-{[2-(3,4-dimethoxyphenyl)ethyl]amino}pyrimidin-4-yl)acetonitrile
1,3-benzothiazol-2-yl(2-{[2-(3-methoxyphenyl)ethyl]amino}pyrimidin-4-yl)acetonitrile
1,3-benzothiazol-2-yl(2-{[2-(2-fluorophenyl)ethyl]amino}pyrimidin-4-yl)acetonitrile
1,3-benzothiazol-2-yl[2-({2-[3-(trifluoromethyl)phenyl]ethyl}amino)pyrimidin-4-yl]aetonitrile
1,3-benzothiazol-2-yl{2-[(2-hydroxy-2-phenylethyl)amino]pyrimidin-4-yl}acetonitrile
1,3-benzothiazol-2-yl{2-[(2-{[3-(trifluoromethyl)pyridin-2-yl]amino}ethyl)amino]-pyrimidin-4-yl}acetonitrile
1,3-benzothiazol-2-yl(2-{[2-(3-chlorophenyl)ethyl]amino}pyrimidin-4-yl)acetonitrile
1,3-benzothiazol-2-yl(2-{[2-(3,4-dichlorophenyl)ethyl]amino}pyrimidin-4-yl)acetonitrile
1,3-benzothiazol-2-yl(2-{[2-(4-methoxyphenyl)ethyl]amino}pyrimidin-4-yl)acetonitrile
1,3-benzothiazol-2-yl(2-{[2-(4-methylphenyl)ethyl]amino}pyrimidin-4-yl)acetonitrile
1,3-benzothiazol-2-yl(2-{[2-(3-fluorophenyl)ethyl]amino}pyrimidin-4-yl)acetonitrile
1,3-benzothiazol-2-yl(2-{[2-(4-phenoxyphenyl)ethyl]amino}pyrimidin-4-yl)acetonitrile
1,3-benzothiazol-2-yl(2-{[2-(2-phenoxyphenyl)ethyl]amino}pyrimidin-4-yl)acetonitrile
1,3-benzothiazol-2-yl(2-{[2-(4-bromophenyl)ethyl]amino}pyrimidin-4-yl)acetonitrile
1,3-benzothiazol-2-yl(2-{[2-(4-fluorophenyl)ethyl]amino}pyrimidin-4-yl)acetonitrile
1,3-benzothiazol-2-yl{2-[(2-[1,1′-biphenyl]-4-ylethyl)amino]pyrimidin-4-yl}acetonitrile
1,3-benzothiazol-2-yl{2-[(2-{4-[hydroxy(oxido)amino]phenyl}ethyl)amino]pyrimidin-4-yl}acetonitrile
1,3-benzothiazol-2-yl(2-{[2-(1H-1,2,4-triazol-1-yl)ethyl]amino}pyrimidin-4-yl)acetonitrile
1,3-benzothiazol-2-yl(2-{[3-(1H-pyrazol-1-yl)propyl]amino}pyrimidin-4-yl)acetonitrile
4-[2-({4-[1,3-benzothiazol-2-yl(cyano)methyl]pyrimidin-2-yl}amino)ethyl]benzene-sulfonamide
{2-[(2-pyridin-3-ylethyl)amino]pyrimidin-4-yl}[5-(trifluoromethyl)-1,3-benzothiazol-2-yl]acetonitrile
1,3-benzothiazol-2-yl{2-[(1H-tetraazol-5-ylmethyl)amino]pyrimidin-4-yl}acetonitrile
1,3-benzothiazol-2-yl[2-(benzyloxy)pyrimidin-4-yl]acetonitrile
1,3-benzothiazol-2-yl{2-[(4-pyridin-3-ylbenzyl)oxy]pyrimidin-4-yl}acetonitrile
1,3-benzothiazol-2-yl[2-(pyridin-4-ylmethoxy)pyrimidin-4-yl]acetonitrile
1,3-benzothiazol-2-yl[2-(pyridin-2-ylmethoxy)pyrimidin-4-yl]acetonitrile
1,3-benzothiazol-2-yl[2-(3-pyridin-2-ylpropoxy)pyrimidin-4-yl]acetonitrile
1,3-benzothiazol-2-yl{2-[(4-methoxybenzyl)oxy]pyrimidin-4-yl}acetonitrile
1,3-benzothiazol-2-yl[2-(pyridin-3-ylmethoxy)pyrimidin-4-yl]acetonitrile
1,3-benzothiazol-2-yl{2-[2-(4-methoxyphenyl)ethoxy]pyrimidin-4-yl}acetonitrile
1,3-benzothiazol-2-yl[2-([1,1′-biphenyl]-3-ylmethoxy)pyrimidin-4-yl]acetonitrile
1,3-benzothiazol-2-yl{2-[(3,4,5-trimethoxybenzyl)oxy]pyrimidin-4-yl}acetonitrile
1,3-benzothiazol-2-yl{2-[(3,4-dichlorobenzyl)oxy]pyrimidin-4-yl}acetonitrile
1,3-benzothiazol-2-yl[2-({3-[(dimethylamino)methyl]benzyl}oxy)pyrimidin-4-yl]acetonitrile
1,3-benzothiazol-2-yl{2-[(1-oxidopyridin-3-yl)methoxy]pyrimidin-4-yl}acetonitrile
1,3-benzothiazol-2-yl(2-{[4-(morpholin-4-ylmethyl)benzyl]oxy}pyrimidin-4-yl)acetonitrile
1,3-benzothiazol-2-yl{2-[(4-pyridin-2-ylbenzyl)oxy]pyrimidin-4-yl}acetonitrile
1,3-benzothiazol-2-yl(2-{[4-(piperidin-1-ylmethyl)benzyl]oxy}pyrimidin-4-yl)acetonitrile
1,3-benzothiazol-2-yl[2-(4-methoxyphenoxy)pyrimidin-4-yl]acetonitrile
1,3-benzothiazol-2-yl[2-(4-butoxyphenoxy)pyrimidin-4-yl]acetonitrile
{2-[4-(4-acetylpiperazin-1-yl)phenoxy]pyrimidin-4-yl}(1,3-benzothiazol-2-yl)acetonitrile
[2-(4-methoxyphenoxy)pyrimidin-4-yl][5-(trifluoromethyl)-1,3-benzothiazol-2-yl]acetonitrile
N-[2-({4-[1,3-benzothiazol-2-yl(cyano)methyl]pyrimidin-2-yl}amino)ethyl]-4-chlorobenzamide
1,3-benzothiazol-2-yl(2-methoxy-4-pyrimidinyl)acetonitrile
1,3-benzothiazol-2-yl[2-({4-[(4-methylpiperazin-1-yl)methyl]benzyl}oxy)pyrimidin-4-yl]acetonitrile
1,3-benzothiazol-2-yl[2-({4-[(4-benzyl-piperazin-1-yl)methyl]-benzyl}oxy)pyrimidin-4-yl]acetonitrile
1,3-benzothiazol-2-yl(2-{[4-(piperazin-1-ylmethyl)benzyl]oxy}pyrimidin-4-yl)acetonitrile
1,3-benzothiazol-2-yl[2-({4-[(4-formylpiperazin-1-yl)methyl]benzyl}oxy)pyrimidin-4-yl]acetonitrile
[2-({4-[(4-acetylpiperazin-1-yl)methyl]benzyl}oxy)pyrimidin-4-yl](1,3-benzothiazol-2-yl)acetonitrile
(3H-Benzothiazol-2-ylidene)-{2-[4-(4-[1,2,4]oxadiazol-3-ylmethyl-piperazin-1-ylmethyl)-benzyloxy]-pyrimidin-4-yl}-acetonitrile
4-(4-{4-[(3H-Benzothiazol-2-ylidene)-cyano-methyl]-pyrimidin-2-yloxymethyl}-benzyl)-piperazine-1-carboxylic acid methyl ester
2-[4-(4-{4-[(3H-Benzothiazol-2-ylidene)-cyano-methyl]-pyrimidin-2-yloxymethyl}-benzyl)-piperazin-1-yl]-acetamide
(2-{4-[4-(2-Amino-acetyl)-piperazin-1-ylmethyl]-benzyloxy}-pyrimidin-4-yl)-(3H-benzothiazol-2-ylidene)-acetonitrile
[4-(4-{4-[(3H-Benzothiazol-2-ylidene)-cyano-methyl]-pyrimidin-2-yloxymethyl}-benzyl)-piperazin-1-yl]-acetic acid methyl ester
(3H-Benzothiazol-2-ylidene)-(2-{4-[4-(2-methoxy-ethyl)-piperazin-1-ylmethyl]-benzyloxy}-pyrimidin-4-yl)-acetonitrile
4-(4-{4-[(3H-Benzothiazol-2-ylidene)-cyano-methyl]-pyrimidin-2-yloxymethyl}-benzyl)-piperazine-1-carboxylic acid dimethylamide
(3H-Benzothiazol-2-ylidene)-{2-[4-(4-ethyl-piperazin-1-ylmethyl)-benzyloxy]-pyrimidin-4-yl}-acetonitrile
(3H-Benzothiazol-2-ylidene)-(2-{4-[4-(2-hydroxy-ethyl)-piperazin-1-ylmethyl]-benzyloxy}-pyrimidin-4-yl)-acetonitrile

The compounds of formula (I) may be obtained according to methods described in WO 01/47920.

In a further embodiment, the compounds of formula (I), are of sub-structure (II) and corresponding tautomers thereof.

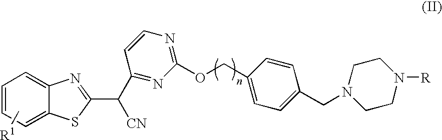

wherein R in formula (II) is selected from the group comprising or consisting of hydrogen, substituted or unsubstituted C₁-C₆-alkyl, substituted or unsubstituted C₁-C₆-alkyl aryl, substituted or unsubstituted heteroaryl, substituted or unsubstituted C₁-C₆-alkyl heteroaryl, substituted or unsubstituted C₂-C₆-alkenyl, substituted or unsubstituted C₂-C₆-alkenyl aryl, substituted or unsubstituted C₂-C₆-alkenyl heteroaryl, substituted or unsubstituted C₂-C₆-alkynyl, substituted or unsubstituted C₂-C₆-alkynyl aryl, substituted or unsubstituted C₂-C₆-alkynyl heteroaryl, substituted or unsubstituted C₃-C₈-cycloalkyl, substituted or unsubstituted heterocycloalkyl, substituted or unsubstituted C₁-C₆-alkyl cycloalkyl, substituted or unsubstituted C₁-C₆-alkyl heterocycloalkyl, substituted or unsubstituted C₁-C₆-alkyl carboxy, acyl, substituted or unsubstituted C₁-C₆-alkyl acyl, acyloxy, substituted or unsubstituted C₁-C₆-alkyl acyloxy, substituted or unsubstituted C₁-C₆-alkyl alkoxy, alkoxycarbonyl, substituted or unsubstituted C₁-C₆-alkyl alkoxycarbonyl, aminocarbonyl, substituted or unsubstituted C₁-C₆-alkyl aminocarbonyl, acylamino, substituted or unsubstituted C₁-C₆-alkyl acylamino, ureido, substituted or unsubstituted C₁-C₆-alkyl ureido, amino, substituted or unsubstituted C₁-C₆-alkyl amino, sulfonyloxy, substituted or unsubstituted C₁-C₆-alkyl sulfonyloxy, sulfonyl, substituted or unsubstituted C₁-C₆-alkyl sulfonyl, sulfinyl, substituted or unsubstituted C₁-C₆-alkyl sulfinyl, sulfanyl, substituted or unsubstituted C₁-C₆-alkyl sulfanyl, sulfonylamino, substituted or unsubstituted C₁-C₆-alkyl sulfonylamino.

R¹ is selected from the group comprising or consisting of H, halogen, cyano, nitro, amino, substituted or unsubstituted C₁-C₆-alkyl, in particular C₁-C₃ alkyl, like methyl or ethyl or —CF₃, substituted or unsubstituted C₂-C₆-alkenyl, substituted or unsubstituted C₂-C₆-alkynyl, substituted or unsubstituted C₁-C₆-alkyl-aryl, substituted or unsubstituted aryl or substituted or unsubstituted heteroaryl, substituted or unsubstituted C₁-C₆-alkyl-heteroaryl, —C(O)—OR²—C(O)—R², —C(O)—NR²R², —(SO₂)R² with

R² and R^{2′} being independently selected from the group comprising or consisting of hydrogen, unsubstituted or substituted C₁-C₆ alkyl, unsubstituted or substituted C₂-C₆ alkenyl, unsubstituted or substituted C₂-C₆ alkynyl, unsubstituted or substituted aryl, unsubstituted or substituted heteroaryl, unsubstituted or substituted C₁-C₆-alkyl aryl, unsubstituted or substituted C₁-C₆-alkyl heteroaryl. Preferably R¹ is H n is an integer from 0 to 3, more preferred is 1.

Specific piperazine benzothiazole derivatives according to the present invention are selected from the following group:
1,3-benzothiazol-2-yl[2-({4-[(4-methylpiperazin-1-yl)methyl]benzyl}oxy)pyrimidin-4-yl]acetonitrile
1,3-benzothiazol-2-yl[2-({4-[(4-benzyl-piperazin-1-yl)methyl]-benzyl}oxy)pyrimidin-4-yl]acetonitrile
1,3-benzothiazol-2-yl(2-{[4-(piperazin-1-ylmethyl)benzyl]oxy}pyrimidin-4-yl)acetonitrile
1,3-benzothiazol-2-yl[2-({4-[(4-formylpiperazin-1-yl)methyl]benzyl}oxy)pyrimidin-4-yl]acetonitrile
[2-({4-[(4-acetylpiperazin-1-yl)methyl]benzyl}oxy)pyrimidin-4-yl](1,3-benzothiazol-2-yl)acetonitrile
(3H-Benzothiazol-2-ylidene)-{2-[4-(4-[1,2,4]oxadiazol-3-ylmethyl-piperazin-1-ylmethyl)-benzyloxy]-pyrimidin-4-yl}-acetonitrile
4-(4-{4-[(3H-Benzothiazol-2-ylidene)-cyano-methyl]-pyrimidin-2-yloxymethyl}-benzyl)-piperazine-1-carboxylic acid methyl ester
2-[4-(4-{4-[(3H-Benzothiazol-2-ylidene)-cyano-methyl]-pyrimidin-2-yloxymethyl}-benzyl)-piperazin-1-yl]-acetamide
(2-{4-[4-(2-Amino-acetyl)-piperazin-1-ylmethyl]-benzyloxy}-pyrimidin-4-yl)-(3H-benzothiazol-2-ylidene)-acetonitrile
[4-(4-{4-[(3H-Benzothiazol-2-ylidene)-cyano-methyl]-pyrimidin-2-yloxymethyl}-benzyl)-piperazin-1-yl]-acetic acid methyl ester
(3H-Benzothiazol-2-ylidene)-(2-{4-[4-(2-methoxy-ethyl)-piperazin-1-ylmethyl]-benzyloxy}-pyrimidin-4-yl)-acetonitrile
4-(4-{4-[(3H-Benzothiazol-2-ylidene)-cyano-methyl]-pyrimidin-2-yloxymethyl}-benzyl)-piperazine-1-carboxylic acid dimethylamide
(3H-Benzothiazol-2-ylidene)-{2-[4-(4-ethyl-piperazin-1-ylmethyl)-benzyloxy]-pyrimidin-4-yl}-acetonitrile
(3H-Benzothiazol-2-ylidene)-(2-{4-[4-(2-hydroxy-ethyl)-piperazin-1-ylmethyl]-benzyloxy}-pyrimidin-4-yl)-acetonitrile.

The compounds of formula (II) may be obtained according to the methods described in WO 03/091249.

In a further embodiment the JNK inhibitors may have the formula (III):

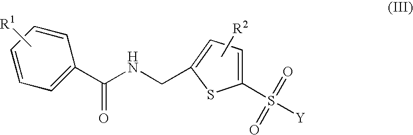

Y is an unsubstituted or a substituted 4-12-membered saturated cyclic or bicyclic alkyl ring containing at least one nitrogen atom (heterocycle), whereby one nitrogen atom within said ring is forming a bond with the sulfonyl group of formula III, thus providing a sulfonamide.

R¹ is selected from the group comprising or consisting of hydrogen, unsubstituted or a substituted C₁-C₆-alkoxy, unsubstituted or a substituted C₁-C₆-alkyl, unsubstituted or a substituted C₂-C₆-alkenyl, unsubstituted or a substituted C₂-C₆-alkynyl, amino, sulfanyl, sulfinyl, sulfonyl, sulfonyloxy, sulfonamide, acylamino, aminocarbonyl, unsubstituted or a substituted C₁-C₆ alkoxycarbonyl, unsubstituted or a substituted aryl, unsubstituted or a substituted heteroaryl, carboxy, cyano, halogen, hydroxy, nitro, hydrazide.

More specifically, R¹ is selected from the group consisting of hydrogen, halogen (e.g. chlorine), C₁-C₆ alkyl (e.g. methyl or ethyl) or C₁-C₆ alkoxy (e.g. methoxy or ethoxy). Most preferred is halogen, in particular chlorine.

R² is selected from the group comprising or consisting of hydrogen, COOR³—CONR³R³, OH, a C₁-C₄ alkyl substituted with an OH or amino group, a hydrazido carbonyl group, a sulfate, a sulfonate, an amine or an ammonium salt. Thereby, R³, R^{3′} are independently selected from the group consisting of H, C₁-C₆-alkyl, C₂-C₆-alkenyl, aryl, heteroaryl, aryl-C₁-C₆-alkyl, heteroaryl-C₁-C₆-alkyl.

According to one embodiment the cyclic amines Y have either of the general formulae (a) to (d):

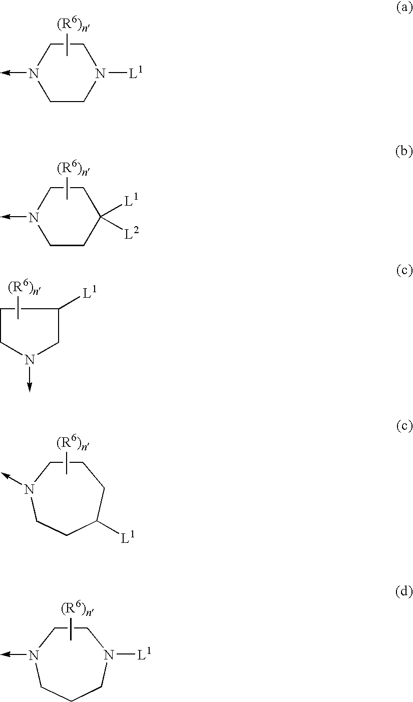

Thereby, L¹ and L² are independently selected from each other from the group consisting of unsubstituted or a substituted C₁-C₆-alkyl, unsubstituted or a substituted C₂-C₆-alkenyl, unsubstituted or a substituted C₂-C₆-alkynyl, unsubstituted or a substituted C₄-C₈-cycloalkyl optionally containing 1-3 heteroatoms and optionally fused with aryl or heteroaryl.

Alternatively, L¹ and L² are independently selected from the group consisting of unsubstituted or a substituted aryl, unsubstituted or a substituted heteroaryl, unsubstituted or a substituted aryl-C₁-C₆-alkyl, unsubstituted or a substituted heteroaryl-C₁-C₆-alkyl, —C(O)—OR³, —C(O)—R³—C(O)—NR^{3′}R³—NR^{3′}R³—NR^{3′}C(O)R³, —NR^{3′}C(O)NR^{3′}R³, —(SO)R³—(SO₂)R³—NSO₂R³—SO₂NR^{3′}R³.

Alternatively, L¹ and L² taken together may form a 4-8-membered, unsubstituted or a substituted saturated cyclic alkyl or heteroalkyl ring.

R³, R^{3′} are independently selected from the group consisting of H, unsubstituted or a substituted C₁-C₆-alkyl, unsubstituted or a substituted C₂-C₆-alkenyl, unsubstituted or a substituted aryl, unsubstituted or a substituted heteroaryl, unsubstituted or a substituted aryl-C₁-C₆-alkyl, unsubstituted or a substituted heteroaryl-C₁-C₆-alkyl.

R⁶ is selected from the group consisting of hydrogen, unsubstituted or a substituted C₁-C₆-alkyl, C₁-C₆-alkoxy, OH, halogen, nitro, cyano, sulfonyl, oxo (═O), and

n′ is an integer from 0 to 4, preferably 1 or 2. In one embodiment R⁶ is hydrogen.

In a further specific embodiment R⁶ is H, L² is H, L¹ is —NR^{3′}R³; where at least one of R^{3′} and R³ is not hydrogen, but a substituent selected from the group consisting of straight or branched C₄-C₁₈-alkyl, aryl-C₁-C₁₈-alkyl, heteroaryl-C₂-C₁₈-alkyl, C₁-C₁₄-alkyl substituted with a C₃-C₁₂-cycloalkyl or -bicyclo or -tricyloalkyl, and whereby said alkyl chain may contain 1-3 O or S atoms.

In a more specific embodiment L¹ is —NHR³; where R³ is a straight or branched C₄-C₁₂-alkyl, preferably a C₆-C₁₂-alkyl, optionally substituted with a cyclohexyl group or a benzyl group.

In an even more specific embodiment Y is a piperidine group

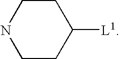

L¹ is —NHR³; where R³ is a straight or branched C₄-C₁₂-alkyl, preferably a C₈-C₁₂-alkyl, or a benzyl group.

Specific examples of compounds of formula (III) include the following:
4-chloro-N-[5-(piperazine-1-sulfonyl)-thiophen-2-yl-methyl]-benzamide
4-Chloro-N-{5-[4-(3-trifluoromethanesulfonyl-phenylamino)-piperidine-1-sulfonyl]-thiophen-2-ylmethyl}-benzamide
4-chloro-N-({5-[(4-pyridin-2-ylpiperazin-1-yl)sulfonyl]thien-2-yl}methyl)benzamide
4-chloro-N-[(5-{[4-(4-fluorobenzoyl)piperidin-1-yl]sulfonyl}thien-2-yl)methyl]benzamide
4-chloro-N-{[5-({4-[4-(trifluoromethyl)phenyl]piperazin-1-yl}sulfonyl)thien-2-yl]methyl}benzamide
4-chloro-N-({5-[(4-{2-nitrophenyl}piperazin-1-yl)sulfonyl]thien-2-yl}methyl)benzamide
4-chloro-N-({5-[(4-{4-nitrophenyl}piperazin-1-yl)sulfonyl]thien-2-yl}methyl)benzamide
4-chloro-N-[(5-{[4-(2-furoyl)piperazin-1-yl]sulfonyl}thien-2-yl)methyl]benzamide
4-chloro-N-[(5-{[4-(4-hydroxyphenyl)piperazin-1-yl]sulfonyl}thien-2-yl)methyl]benzamide
4-chloro-N-[(5-{[4-(2-oxo-2-pyrrolidin-1-ylethyl)piperazin-1-yl]sulfonyl}thien-2-yl)methyl]benzamide
4-chloro-N-[(5-{[4-(2-morpholin-4-yl-2-oxoethyl)piperazin-1-yl]sulfonyl}thien-2-yl)methyl]benzamide
4-chloro-N-[(5-{[4-(pyridin-4-ylmethyl)piperazin-1-yl]sulfonyl}thien-2-yl)methyl]benzamide
4-chloro-N-[(5-{[4-(2-thien-2-ylethyl)piperazin-1-yl]sulfonyl}thien-2-yl)methyl]benzamide
4-chloro-N-[(5-{[4-(3,5-dimethoxyphenyl)piperazin-1-yl]sulfonyl}thien-2-yl)methyl]benzamide
4-chloro-N-[(5-{[4-(cyclohexylmethyl)piperazin-1-yl]sulfonyl}thien-2-yl)methyl]benzamide
4-chloro-N-[(5-{[4-(2-methoxyphenyl)piperazin-1-yl]sulfonyl}thien-2-yl)methyl]benzamide
N-({5-[(4-benzylpiperazin-1-yl)sulfonyl]thien-2-yl}methyl)-4-chlorobenzamide
4-chloro-N-[(5-{[4-(2-phenylethyl)piperazin-1-yl]sulfonyl}thien-2-yl)methyl]benzamide
4-chloro-N-[(5-{[4-(4-fluorobenzyl)piperazin-1-yl]sulfonyl}thien-2-yl)methyl]benzamide
4-chloro-N-[(5-{[4-(2-cyanophenyl)piperazin-1-yl]sulfonyl}thien-2-yl)methyl]benzamide
4-chloro-N-{[5-({4-[4-chloro-3-(trifluoromethyl)phenyl]piperazin-1-yl}sulfonyl)thien-2-yl]methyl}benzamide
4-chloro-N-[(5-{[4-(3-piperidin-1-ylpropyl)piperazin-1-yl]sulfonyl}thien-2-yl)methyl]benzamide
4-chloro-N-({5-[(4-{4-chloro-2-nitrophenyl}piperazin-1-yl)sulfonyl]thien-2-yl}methyl)benzamide
4-chloro-N-[(5-{[4-(6-methylpyridin-2-yl)piperazin-1-yl]sulfonyl}thien-2-yl)methyl]benzamide
4-chloro-N-({5-[(4-hydroxy-4-phenylpiperidin-1-yl)sulfonyl]thien-2-yl}methyl)benzamide
N-({5-[(4-benzylpiperidin-1-yl)sulfonyl]thien-2-yl}methyl)-4-chlorobenzamide
4-chloro-N-[(5-{[4-(2-oxo-2,3-dihydro-1H-benzimidazol-1-yl)piperidin-1-yl]sulfonyl}thien-2-yl)methyl]benzamide
N-({5-[(4-benzylpiperidin-1-yl)sulfonyl]thien-2-yl}methyl)-4-chlorobenzamide
4-chloro-N-({5-[(4-oxo-1-phenyl-1,3,8-triazaspiro[4.5]dec-8-yl)sulfonyl]thien-2-yl}methyl)benzamide
4-chloro-N-{[5-({4-[2-(methylanilino)-2-oxoethyl]piperazin-1-yl}sulfonyl)thien-2-yl]methyl}benzamide
4-chloro-N-{[5-({4-[hydroxy(diphenyl)methyl]piperidin-1-yl}sulfonyl)thien-2-yl]methyl}benzamide
4-chloro-N-[(5-{[4-(3-cyanopyrazin-2-yl)piperazin-1-yl]sulfonyl}thien-2-yl)methyl]benzamide
4-chloro-N-({5-[(4-{5-nitropyridin-2-yl}piperazin-1-yl)sulfonyl]thien-2-yl}methyl)benzamide
4-chloro-N-{[5-({4-[3-chloro-5-(trifluoromethyl)pyridin-2-yl]piperazin-1-yl}sulfonyl)thien-2-yl]methyl}benzamide
4-chloro-N-{[5-({4-[5-(trifluoromethyl)pyridin-2-yl]piperazin-1-yl}sulfonyl)thien-2-yl]methyl}benzamide
4-chloro-N-{[5-({4-[3-(trifluoromethyl)pyridin-2-yl]piperazin-1-yl}sulfonyl)thien-2-yl]methyl}benzamide
4-chloro-N-[(5-{[4-(2,4-difluorobenzoyl)piperidin-1-yl]sulfonyl}thien-2-yl)methyl]benzamide
methyl 5-{4-[(5-{[(4-chlorobenzoyl)amino]methyl}thien-2-yl)sulfonyl]piperazin-1-yl}-7-(trifluoromethyl)thieno[3,2-b]pyridine-3-carboxylate
ethyl 2-{4-[(5-{[(4-chlorobenzoyl)amino]methyl}thien-2-yl)sulfonyl]piperazin-1-yl}-5-cyano-6-methylnicotinate
4-chloro-N-{[5-({4-[5-cyano-4,6-bis(dimethylamino)pyridin-2-yl]piperazin-1-yl}sulfonyl)thien-2-yl]methyl}benzamide
4-chloro-N-{[5-({4-[6-methyl-2-(trifluoromethyl)quinolin-4-yl]piperazin-1-yl}sulfonyl)thien-2-yl]methyl}benzamide
tert-butyl 4-[(5-{[(4-chlorobenzoyl)amino]methyl}thien-2-yl)sulfonyl]piperazine-1-carboxylate
2-{4-[(5-{[(4-chlorobenzoyl)amino]methyl}thien-2-yl)sulfonyl]piperazin-1-yl}-8-ethyl-5-oxo-5,8-dihydropyrido[2,3-d]pyrimidine-6-carboxylic acid
7-{4-[(5-{[(4-chlorobenzoyl)amino]methyl}thien-2-yl)sulfonyl]piperazin-1-yl}-1-ethyl-6-fluoro-4-oxo-1,4-dihydro[1,8]naphthyridine-3-carboxylic acid
7-{4-[(5-{[(4-chlorobenzoyl)amino]methyl}thien-2-yl)sulfonyl]piperazin-1-yl}-1-ethyl-6-fluoro-4-oxo-1,4-dihydroquinoline-3-carboxylic acid
4-chloro-N-[(5-{[4-(2,3-dihydro-1,4-benzodioxin-2-ylcarbonyl)piperazin-1-yl]sulfonyl}thien-2-yl)methyl]benzamide
4-chloro-N-{[5-({4-[(2E)-3-phenylprop-2-enyl]piperazin-1-yl}sulfonyl)thien-2-yl]methyl}benzamide
4-chloro-N-[(5-{[4-(3-phenylpropyl)piperazin-1-yl]sulfonyl}thien-2-yl)methyl]benzamide
4-chloro-N-[(5-{[4-(3,4,5-trimethoxyphenyl)piperazin-1-yl]sulfonyl}thien-2-yl)methyl]benzamide
N-[(5-{[4-(4-tert-butylbenzyl)piperazin-1-yl]sulfonyl}thien-2-yl)methyl]-4-chlorobenzamide
4-chloro-N-[(5-{[4-(4-fluorophenyl)piperazin-1-yl]sulfonyl}thien-2-yl)methyl]benzamide
4-chloro-N-[(5-{[4-(2-hydroxyphenyl)piperazin-1-yl]sulfonyl}thien-2-yl)methyl]benzamide
4-chloro-N-{[5-({4-[4-(trifluoromethyl)pyridin-2-yl]piperazin-1-yl}sulfonyl)thien-2-yl]methyl}benzamide
4-chloro-N-[(5-{[4-(5-cyanopyridin-2-yl)piperazin-1-yl]sulfonyl}thien-2-yl)methyl]benzamide tert-butyl 1-[(5-{[(4-chlorobenzoyl)amino]methyl}thien-2-yl)sulfonyl]piperidin-4-ylcarbamate
4-chloro-N-({5-[(4-phenylpiperazin-1-yl)sulfonyl]thien-2-yl}methyl)benzamide
4-chloro-N-{[5-(piperidin-1-ylsulfonyl)thien-2-yl]methyl}benzamide
4-chloro-N-[(5-{[4-(1-naphthyl)piperazin-1-yl]sulfonyl}thien-2-yl)methyl]benzamide
4-chloro-N-[(5-{[4-(3,4-dichlorophenyl)piperazin-1-yl]sulfonyl}thien-2-yl)methyl]benzamide
4-chloro-N-{[5-({4-[3-(trifluoromethyl)phenyl]piperazin-1-yl}sulfonyl)thien-2-yl]methyl}benzamide
4-chloro-N-{[5-({3-hydroxy-4-[3-(trifluoromethyl)phenyl]piperidin-1-yl}sulfonyl)thien-2-yl]methyl}benzamide
4-chloro-N-[(5-{[4-(2-methylphenyl)piperazin-1-yl]sulfonyl}thien-2-yl)methyl]benzamide
N-[(5-{[(1R,4R)-5-benzyl-2,5-diazabicyclo[2.2.1]hept-2-yl]sulfonyl}thien-2-yl)methyl]-4-chlorobenzamide
N-[(5-{[4-(benzyloxy)piperidin-1-yl]sulfonyl}thien-2-yl)methyl]-4-chlorobenzamide
4-chloro-N-[(5-{[4-(2-chlorodibenzo[b,f][1,4]oxazepin-11-yl)piperazin-1-yl]sulfonyl}thien-2-yl)methyl]benzamide
N-(4-chlorophenyl)-2-(5-{[4-(2-oxo-2,3-dihydro-1H-benzimidazol-1-yl)piperidin-1-yl]sulfonyl}thien-2-yl)acetamide
4-chloro-N-({5-[(4-hydroxypiperidin-1-yl)sulfonyl]thien-2-yl}methyl)benzamide
N-[(5-{[4-(4-acetylphenyl)piperazin-1-yl]sulfonyl}thien-2-yl)methyl]-4-chlorobenzamide
4-chloro-N-[(5-{[4-(3,5-dichloropyridin-4-yl)piperazin-1-yl]sulfonyl}thien-2-yl)methyl]benzamide
4-chloro-N-[(5-{[4-(3-methoxyphenyl)piperazin-1-yl]sulfonyl}thien-2-yl)methyl]benzamide
N-({5-[(4-benzyl-4-hydroxypiperidin-1-yl)sulfonyl]thien-2-yl}methyl)-4-chlorobenzamide
N-{[5-({4-[(2-tert-butyl-1H-indol-5-yl)amino]piperidin-1-yl}sulfonyl)thien-2-yl]methyl}-4-chlorobenzamide
4-chloro-N-{[5-({4-[(phenylacetyl)amino]piperidin-1-yl}sulfonyl)thien-2-yl]methyl}benzamide
4-chloro-N-[(5-{[4-(tetrahydrofuran-2-ylcarbonyl)piperazin-1-yl]sulfonyl}thien-2-yl)methyl]benzamide
4-chloro-N-[(5-{[4-(6-chloropyridin-2-yl)piperazin-1-yl]sulfonyl}thien-2-yl)methyl]benzamide
4-chloro-N-[(5-{[4-(4-chlorophenyl)piperazin-1-yl]sulfonyl}thien-2-yl)methyl]benzamide
N-[(5-{[4-(2H-1,2,3-benzotriazol-2-yl)piperidin-1-yl]sulfonyl}thien-2-yl)methyl]-4-chlorobenzamide
4-chloro-N-[(5-{[4-(4-chlorobenzoyl)piperidin-1-yl]sulfonyl}thien-2-yl)methyl]benzamide
4-chloro-N-({5-[(4-phenoxypiperidin-1-yl)sulfonyl]thien-2-yl}methyl)benzamide
N-{[5-({4-[benzyl(methyl)amino]piperidin-1-yl}sulfonyl)thien-2-yl]methyl}-4-chlorobenzamide
4-chloro-N-{[5-({4-[3-(2,4-dichlorophenyl)-1H-pyrazol-5-yl]piperidin-1-yl}sulfonyl)thien-2-yl]methyl}benzamide
4-chloro-N-[(5-{[4-(5-thien-2-yl-1H-pyrazol-3-yl)piperidin-1-yl]sulfonyl}thien-2-yl)methyl]benzamide
4-chloro-N-[(5-{[4-(2,3,4,5,6-pentamethylbenzoyl)piperidin-1-yl]sulfonyl}thien-2-yl)methyl]benzamide
4-chloro-N-[(5-{[4-(phenylacetyl)-1,4-diazepan-1-yl]sulfonyl}thien-2-yl)methyl]benzamide
4-chloro-N-{[5-({4-[5-(4-methoxyphenyl)-1H-pyrazol-3-yl]piperidin-1-yl}sulfonyl)thien-2-yl]methyl}benzamide
N-({5-[(4-anilinopiperidin-1-yl)sulfonyl]thien-2-yl}methyl)-4-chlorobenzamide
4-chloro-N-[(5-{[4-(3-phenyl-1,2,4-thiadiazol-5-yl)piperazin-1-yl]sulfonyl}thien-2-yl)methyl]benzamide
4-chloro-N-[(5-{[4-(2-phenylethyl)piperidin-1-yl]sulfonyl}thien-2-yl)methyl]benzamide
4-chloro-N-({5-[(4-heptylpiperazin-1-yl)sulfonyl]thien-2-yl}methyl)benzamide
4-chloro-N-({5-[(4-octylpiperazin-1-yl)sulfonyl]thien-2-yl}methyl)benzamide
N-[(5-{[4-(1H-1,2,3-benzotriazol-1-yl)piperidin-1-yl]sulfonyl}thien-2-yl)methyl]-4-chlorobenzamide
2-(5-{[4-(1H-1,2,3-benzotriazol-1-yl)piperidin-1-yl]sulfonyl}thien-2-yl)-N-(4-chlorophenyl)acetamide
2-{1-[(5-{[(4-chlorobenzoyl)amino]methyl}thien-2-yl)sulfonyl]piperidin-4-yl}-2H-1,2,3-benzotriazole-5-carboxylic acid
4-chloro-N-[(5-{[4-(5-chloro-1H-1,2,3-benzotriazol-1-yl)piperidin-1-yl]sulfonyl}thien-2-yl)methyl]benzamide
methyl 1-{1-[(5-{[(4-chlorobenzoyl)amino]methyl}thien-2-yl)sulfonyl]piperidin-4-yl}-1H-1,2,3-benzotriazole-5-carboxylate
methyl 1-{1-[(5-{[(4-chlorobenzoyl)amino]methyl}thien-2-yl)sulfonyl]piperidin-4-yl}-1H-1,2,3-benzotriazole-6-carboxylate
methyl 2-{1-[(5-{[(4-chlorobenzoyl)amino]methyl}thien-2-yl)sulfonyl]piperidin-4-yl}-2H-1,2,3-benzotriazole-5-carboxylate
4-chloro-N-[(5-{[4-(6-chloro-1H-1,2,3-benzotriazol-1-yl)piperidin-1-yl]sulfonyl}thien-2-yl)methyl]benzamide
4-chloro-N-{[5-({4-[5-(trifluoromethyl)-1H-1,2,3-benzotriazol-1-yl]piperidin-1-yl}sulfonyl)thien-2-yl]methyl}benzamide
N-[(5-{[4-(7-aza-1H-benzimidazol-1-yl)piperidin-1-yl]sulfonyl}thien-2-yl)methyl]-4-chlorobenzamide
1-{1-[(5-{[(4-chlorobenzoyl)amino]methyl}thien-2-yl)sulfonyl]piperidin-4-yl}-1H-1,2,3-benzotriazole-5-carboxylic acid
1-{1-[(5-{[(4-chlorobenzoyl)amino]methyl}thien-2-yl)sulfonyl]piperidin-4-yl}-1H-1,2,3-benzotriazole-6-carboxylic acid
N-[(5-{[4-(2-amino-9H-purin-9-yl)piperidin-1-yl]sulfonyl}thien-2-yl)methyl]-4-chlorobenzamide
4-chloro-N-[(5-{[4-(9H-purin-9-yl)piperidin-1-yl]sulfonyl}thien-2-yl)methyl]benzamide
N-[(5-{[4-(6-amino-9H-purin-9-yl)piperidin-1-yl]sulfonyl}thien-2-yl)methyl]-4-chlorobenzamide
4-chloro-N-({5-[(4-{6-nitro-1H-benzimidazol-1-yl}piperidin-1-yl)sulfonyl]thien-2-yl}methyl)benzamide
4-chloro-N-({5-[(4-{5-nitro-1H-benzimidazol-1-yl}piperidin-1-yl)sulfonyl]thien-2-yl}methyl)benzamide
4-chloro-N-[(5-{[4-(2H-1,2,3-triazol-2-yl)piperidin-1-yl]sulfonyl}thien-2-yl)methyl]benzamide
N-[(5-{[4-(1H-benzimidazol-1-yl)piperidin-1-yl]sulfonyl}thien-2-yl)methyl]-4-chlorobenzamide
4-chloro-N-{[5-({4-[3-propylanilino]piperidin-1-yl}sulfonyl)thien-2-yl]methyl}benzamide
4-chloro-N-{[5-({4-[3-(trifluoromethyl)anilino]piperidin-1-yl}sulfonyl)thien-2-yl]methyl}benzamide
4-chloro-N-{[5-({4-[3-(dimethylamino)anilino]piperidin-1-yl}sulfonyl)thien-2-yl]methyl}benzamide
methyl 3-({1-[(5-{[(4-chlorobenzoyl)amino]-methyl}thien-2-yl)sulfonyl]-piperidin-4-yl}amino)-benzoate
4-chloro-N-{[5-({4-[3-(methylsulfanyl)anilino]piperidin-1-yl}sulfonyl)thien-2-yl]methyl}benzamide
4-chloro-N-({5-[(4-{3-nitroanilino}piperidin-1-yl)sulfonyl]thien-2-yl}methyl)benzamide
4-chloro-N-[(5-{[4-(2-methoxyanilino)piperidin-1-yl]sulfonyl}thien-2-yl)methyl]benzamide
3-({1-[(5-{[(4-chlorobenzoyl)amino]methyl}thien-2-yl)sulfonyl]piperidin-4-yl}amino)benzamide
4-chloro-N-{[5-({4-[2-(trifluoromethyl)anilino]piperidin-1-yl}sulfonyl)thien-2-yl]methyl}benzamide
4-chloro-N-({5-[(4-{2-nitro-4-[(trifluoromethyl)sulfonyl]anilino}piperidin-1-yl)sulfonyl]thien-2-yl}methyl)benzamide
4-chloro-N-[(5-{[4-(4-chloroanilino)piperidin-1-yl]sulfonyl}thien-2-yl)methyl]benzamide
4-chloro-N-{[5-({4-[4-(trifluoromethyl)anilino]piperidin-1-yl}sulfonyl)thien-2-yl]methyl}benzamide
4-chloro-N-({5-[(4-{4-[(trifluoromethyl)sulfonyl]anilino}piperidin-1-yl)sulfonyl]thien-2-yl}methyl)benzamide
4-chloro-N-({5-[(4-{2-nitroanilino}piperidin-1-yl)sulfonyl]thien-2-yl}methyl)benzamide
N-{[5-({4-[4-(aminocarbonyl)anilino]piperidin-1-yl}sulfonyl)thien-2-yl]methyl}-4-chlorobenzamide
4-chloro-N-{[5-({4-[4-(1,3-dithiolan-2-yl)anilino]piperidin-1-yl}sulfonyl)thien-2-yl]methyl}benzamide
N-[(5-{[4-(3-chloroanilino)piperidin-1-yl]sulfonyl}thien-2-yl)methyl]-3-nitrobenzamide
4-chloro-N-[(5-{[4-(3-chloroanilino)piperidin-1-yl]sulfonyl}thien-2-yl)methyl]benzamide
4-chloro-N-[(5-{[4-(3-methoxyanilino)piperidin-1-yl]sulfonyl}thien-2-yl)methyl]benzamide
4-chloro-N-{[5-({4-[3-(methylsulfonyl)anilino]piperidin-1-yl}sulfonyl)thien-2-yl]methyl}benzamide
N-({5-[(4-{3-[amino(imino)methyl]anilino}piperidin-1-yl)sulfonyl]thien-2-yl}methyl)-4-chlorobenzamide
4-chloro-N-({5-[(4-{3-[(2-hydroxyethyl)sulfonyl]anilino}piperidin-1-yl)sulfonyl]thien-2-yl}methyl)benzamide
N-[(5-{[4-(2-aminoanilino)piperidin-1-yl]sulfonyl}thien-2-yl)methyl]-4-chlorobenzamide
4-chloro-N-[(5-{[4-(2-hydroxyanilino)piperidin-1-yl]sulfonyl}thien-2-yl)methyl]benzamide
4-chloro-N-[(5-{[4-(4-hydroxyanilino)piperidin-1-yl]sulfonyl}thien-2-yl)methyl]benzamide
4-chloro-N-({5-[(4-{3-[(trifluoromethyl)sulfanyl]anilino}piperidin-1-yl)sulfonyl]thien-2-yl}methyl)benzamide
4-chloro-N-[(5-{[4-(3-toluidino)piperidin-1-yl]sulfonyl}thien-2-yl)methyl]benzamide
4-chloro-N-({5-[(4-{[3-chloro-5-(trifluoromethyl)pyridin-2-yl]amino}piperidin-1-yl)sulfonyl]thien-2-yl}methyl)benzamide
4-chloro-N-{[5-({4-[3-(1,3-oxazol-5-yl)anilino]piperidin-1-yl}sulfonyl)thien-2-yl]methyl}benzamide
N-[(5-{[4-(3-tert-butylanilino)piperidin-1-yl]sulfonyl}thien-2-yl)methyl]-4-chlorobenzamide
4-chloro-N-[(5-{[4-(2-propylanilino)piperidin-1-yl]sulfonyl}thien-2-yl)methyl]benzamide
4-chloro-N-{[5-({4-[(2,2-dioxido-1,3-dihydro-2-benzothien-5-yl)amino]piperidin-1-yl}sulfonyl)thien-2-yl]methyl}benzamide
4-chloro-N-[(5-{[4-(2,3-dihydro-1H-inden-5-ylamino)piperidin-1-yl]sulfonyl}thien-2-yl)methyl]benzamide
4-chloro-N-[(5-{[4-(4-propylanilino)piperidin-1-yl]sulfonyl}thien-2-yl)methyl]benzamide
4-chloro-N-[(5-{[4-({3-nitropyridin-2-yl}amino)piperidin-1-yl]sulfonyl}thien-2-yl)methyl]benzamide
N-{[5-({4-[(3-aminopyridin-2-yl)amino]piperidin-1-yl}sulfonyl)thien-2-yl]methyl}-4-chlorobenzamide
N-[(5-{[4-([1,1′-biphenyl]-3-ylamino)piperidin-1-yl]sulfonyl}thien-2-yl)methyl]-4-chlorobenzamide
N-[(5-{[4-(3-benzylanilino)piperidin-1-yl]sulfonyl}thien-2-yl)methyl]-4-chlorobenzamide
4-chloro-N-[(5-{[4-(pyrimidin-2-ylamino)piperidin-1-yl]sulfonyl}thien-2-yl)methyl]benzamide
4-chloro-N-{[5-({4-[4-(morpholin-4-ylsulfonyl)anilino]piperidin-1-yl}sulfonyl)thien-2-yl]methyl}benzamide
4-chloro-N-({5-[(4-{[4-(trifluoromethyl)pyrimidin-2-yl]amino}piperidin-1-yl)sulfonyl]thien-2-yl}methyl)benzamide
4-chloro-N-[(5-{[4-(3-cyclohexyl-4-hydroxyanilino)piperidin-1-yl]sulfonyl}thien-2-yl)methyl]benzamide
N-({5-[(4-{3-[(butylamino)sulfonyl]anilino}piperidin-1-yl)sulfonyl]thien-2-yl}methyl)-4-chlorobenzamide
4-chloro-N-[(5-{[4-(3-ethylanilino)piperidin-1-yl]sulfonyl}thien-2-yl)methyl]benzamide
4-chloro-N-[(5-{[4-(5,6,7,8-tetrahydronaphthalen-1-ylamino)piperidin-1-yl]sulfonyl}thien-2-yl)methyl]benzamide
N-{[5-({4-[3-(aminosulfonyl)anilino]piperidin-1-yl}sulfonyl)thien-2-yl]methyl}-4-chlorobenzamide
4-chloro-N-[(5-{[4-(quinolin-5-ylamino)piperidin-1-yl]sulfonyl}thien-2-yl)methyl]benzamide
4-chloro-N-[(5-{[4-(quinolin-8-ylamino)piperidin-1-yl]sulfonyl}thien-2-yl)methyl]benzamide
4-Chloro-N-[(5-{[4-(3-propylphenoxy)piperidin-1-yl]sulfonyl}thien-2-yl)methyl]benzamide
4-chloro-N-{[5-({4-[(2E)-3-phenylprop-2-enoyl]piperazin-1-yl}sulfonyl)thien-2-yl]methyl}benzamide
4-chloro-N-({5-[(4-{4-nitrobenzoyl}piperazin-1-yl)sulfonyl]thien-2-yl}methyl)benzamide
N-({5-[(4-benzoylpiperazin-1-yl)sulfonyl]thien-2-yl}methyl)-4-chlorobenzamide
4-chloro-N-{[5-({4-[4-(trifluoromethyl)benzoyl]piperazin-1-yl}sulfonyl)thien-2-yl]methyl}benzamide
4-chloro-N-{[5-({4-[4-(dimethylamino)benzoyl]piperazin-1-yl}sulfonyl)thien-2-yl]methyl}benzamide
4-chloro-N-[(5-{[4-(2-fluorobenzoyl)piperazin-1-yl]sulfonyl}thien-2-yl)methyl]benzamide
4-chloro-N-[(5-{[4-(2,6-difluorobenzoyl)piperazin-1-yl]sulfonyl}thien-2-yl)methyl]benzamide
4-chloro-N-[(5-{[4-(3-fluorobenzoyl)piperazin-1-yl]sulfonyl}thien-2-yl)methyl]benzamide
4-chloro-N-[(5-{[4-(2-naphthoyl)piperazin-1-yl]sulfonyl}thien-2-yl)methyl]benzamide
4-chloro-N-[(5-{[4-(1-naphthoyl)piperazin-1-yl]sulfonyl}thien-2-yl)methyl]benzamide
4-chloro-N-({5-[(4-{2-nitrobenzoyl}piperazin-1-yl)sulfonyl]thien-2-yl}methyl)benzamide
4-chloro-N-[(5-{[4-(pyridin-3-ylcarbonyl)piperazin-1-yl]sulfonyl}thien-2-yl)methyl]benzamide
N-[(5-{[4-(2,1,3-benzoxadiazol-5-ylcarbonyl)piperazin-1-yl]sulfonyl}thien-2-yl)methyl]-4-chlorobenzamide
4-chloro-N-[(5-{[4-(2,4-difluorobenzoyl)piperazin-1-yl]sulfonyl}thien-2-yl)methyl]benzamide
4-chloro-N-[(5-{[4-(2,4,6-trifluorobenzoyl)piperazin-1-yl]sulfonyl}thien-2-yl)methyl]benzamide
4-chloro-N-[(5-{[4-(2,6-dichlorobenzoyl)piperazin-1-yl]sulfonyl}thien-2-yl)methyl]benzamide
4-chloro-N-({5-[(4-heptanoylpiperazin-1-yl)sulfonyl]thien-2-yl}methyl)benzamide
4-chloro-N-[(5-{[4-(quinolin-8-ylsulfonyl)piperazin-1-yl]sulfonyl}thien-2-yl)methyl]benzamide
4-nitro-N-({5-[(4-{3-[(trifluoromethyl)sulfonyl]anilino}piperidin-1-yl)sulfonyl]thien-2-yl}methyl)benzamide
N-[(5-{[4-(1H-1,2,3-benzotriazol-1-yl)piperidin-1-yl]sulfonyl}thien-2-yl)methyl]-3-nitrobenzamide
4-nitro-N-({5-[(4-{3-[(trifluoromethyl)sulfonyl]anilino}piperidin-1-yl)sulfonyl]thien-2-yl}methyl)benzamide
N-[(5-{[4-(2,4-difluorobenzoyl)piperidin-1-yl]sulfonyl}thien-2-yl)methyl]-4-nitrobenzamide
N-[(5-{[4-(1H-1,2,3-benzotriazol-1-yl)piperidin-1-yl]sulfonyl}thien-2-yl)methyl]-4-nitrobenzamide
N-[(5-{[4-(1H-1,2,3-benzotriazol-1-yl)piperidin-1-yl]sulfonyl}thien-2-yl)methyl]-3-nitrobenzamide
4-nitro-N-({5-[(4-{3-[(trifluoromethyl)sulfonyl]anilino}piperidin-1-yl)sulfonyl]thien-2-yl}methyl)benzamide
N-[(5-{[4-(2,4-difluorobenzoyl)piperidin-1-yl]sulfonyl}thien-2-yl)methyl]-4-nitrobenzamide
N-[(5-{[4-(1H-1,2,3-benzotriazol-1-yl)piperidin-1-yl]sulfonyl}thien-2-yl)methyl]-4-nitrobenzamide
3-nitro-N-[(5-{[4-(3-methoxyanilino)piperidin-1-yl]sulfonyl}thien-2-yl)methyl]benzamide
3-nitro-N-{[5-({4-[3-(trifluoromethyl)anilino]piperidin-1-yl}sulfonyl)thien-2-yl]methyl}benzamide
N-{[5-({4-[3-(dimethylamino)anilino]piperidin-1-yl}sulfonyl)thien-2-yl]methyl}-3-nitrobenzamide
3-nitro-N-{[5-({4-[3-(methylsulfonyl)anilino]piperidin-1-yl}sulfonyl)thien-2-yl]methyl}benzamide
3-nitro-N-{[5-({4-[3-(methylsulfanyl)anilino]piperidin-1-yl}sulfonyl)thien-2-yl]methyl}benzamide
N-{[5-({4-[3-(aminosulfonyl)anilino]piperidin-1-yl}sulfonyl)thien-2-yl]methyl}-3-nitrobenzamide
methyl 3-{[1-({5-[({3-nitrobenzoyl}amino)methyl]-thien-2-yl}sulfonyl)-piperidin-4-yl]amino}benzoate
N-{[5-({4-[3-(aminocarbonyl)anilino]piperidin-1-yl}sulfonyl)thien-2-yl]methyl}-3-nitrobenzamide
3-nitro-N-({5-[(4-{3-nitroanilino}piperidin-1-yl)sulfonyl]thien-2-yl}methyl)benzamide
3-nitro-N-[(5-{[4-(2-methoxyanilino)piperidin-1-yl]sulfonyl}thien-2-yl)methyl]benzamide
3-nitro-N-{[5-({4-[2-(trifluoromethyl)anilino]piperidin-1-yl}sulfonyl)thien-2-yl]methyl}benzamide
3-nitro-N-({5-[(4-{2-nitroanilino}piperidin-1-yl)sulfonyl]thien-2-yl}methyl)benzamide
N-[(5-{[4-(4-chloroanilino)piperidin-1-yl]sulfonyl}thien-2-yl)methyl]-3-nitrobenzamide
3-nitro-N-{[5-({4-[4-(trifluoromethyl)anilino]piperidin-1-yl}sulfonyl)thien-2-yl]methyl}benzamide
3-nitro-N-({5-[(4-{4-[(trifluoromethyl)sulfonyl]anilino}piperidin-1-yl)sulfonyl]thien-2-yl}methyl)benzamide
N-{[5-({4-[4-(aminocarbonyl)anilino]piperidin-1-yl}sulfonyl)thien-2-yl]methyl}-3-nitrobenzamide
N-[(5-{[4-(3-propylanilino)piperidin-1-yl]sulfonyl}thien-2-yl)methyl]-3-nitrobenzamide
N-[(5-{[4-(3-chloroanilino)piperidin-1-yl]sulfonyl}thien-2-yl)methyl]-4-nitrobenzamide
4-nitro-N-[(5-{[4-(3-methoxyanilino)piperidin-1-yl]sulfonyl}thien-2-yl)methyl]benzamide
4-nitro-N-{[5-({4-[3-(trifluoromethyl)anilino]piperidin-1-yl}sulfonyl)thien-2-yl]methyl}benzamide
N-{[5-({4-[3-(dimethylamino)anilino]piperidin-1-yl}sulfonyl)thien-2-yl]methyl}-4-nitrobenzamide
4-nitro-N-[(5-{[4-(3-propylanilino)piperidin-1-yl]sulfonyl}thien-2-yl)methyl]benzamide
4-nitro-N-{[5-({4-[3-(methylsulfonyl)anilino]piperidin-1-yl}sulfonyl)thien-2-yl]methyl}benzamide
4-nitro-N-{[5-({4-[3-(methylsulfanyl)anilino]piperidin-1-yl}sulfonyl)thien-2-yl]methyl}benzamide
N-{[5-({4-[3-(aminosulfonyl)anilino]piperidin-1-yl}sulfonyl)thien-2-yl]methyl}-4-nitrobenzamide
3-{[1-({5-[({4-nitrobenzoyl}amino)methyl]thien-2-yl}sulfonyl)piperidin-4-yl]amino}benzamide
3-{[1-({5-[({4-nitrobenzoyl}amino)methyl]thien-2-yl}sulfonyl)piperidin-4-yl]amino}benzamide
4-nitro-N-({5-[(4-{3-nitroanilino}piperidin-1-yl)sulfonyl]thien-2-yl}methyl)benzamide
4-nitro-N-[(5-{[4-(2-methoxyanilino)piperidin-1-yl]sulfonyl}thien-2-yl)methyl]benzamide
4-nitro-N-{[5-({4-[2-(trifluoromethyl)anilino]piperidin-1-yl}sulfonyl)thien-2-yl]methyl}benzamide
4-nitro-N-({5-[(4-{2-nitroanilino}piperidin-1-yl)sulfonyl]thien-2-yl}methyl)benzamide
N-[(5-{[4-(4-chloroanilino)piperidin-1-yl]sulfonyl}thien-2-yl)methyl]-4-nitrobenzamide
4-nitro-N-{[5-({4-[4-(trifluoromethyl)anilino]piperidin-1-yl}sulfonyl)thien-2-yl]methyl}benzamide
4-nitro-N-({5-[(4-{4-[(trifluoromethyl)sulfonyl]anilino}piperidin-1-yl)sulfonyl]thien-2-yl}methyl)benzamide
N-{[5-({4-[4-(aminocarbonyl)anilino]piperidin-1-yl}sulfonyl)thien-2-yl]methyl}-4-nitrobenzamide
N-{[5-({4-[4-(1,3-dithiolan-2-yl)anilino]piperidin-1-yl}sulfonyl)thien-2-yl]methyl}-4-nitrobenzamide
N-({5-[(4-{3-[amino(imino)methyl]anilino}piperidin-1-yl)sulfonyl]thien-2-yl}methyl)-3-nitrobenzamide
N-({5-[(4-{3-[(2-hydroxyethyl)sulfonyl]anilino}piperidin-1-yl)sulfonyl]thien-2-yl}methyl)-3-nitrobenzamide
N-({5-[(4-anilinopiperidin-1-yl)sulfonyl]thien-2-yl}methyl)-3-nitrobenzamide
N-({5-[(4-{3-[(2-hydroxyethyl)sulfonyl]anilino}piperidin-1-yl)sulfonyl]thien-2-yl}methyl)-4-nitrobenzamide
N-({5-[(4-anilinopiperidin-1-yl)sulfonyl]thien-2-yl}methyl)-4-nitrobenzamide
N-({5-[(4-{3-[amino(imino)methyl]anilino}piperidin-1-yl)sulfonyl]thien-2-yl}methyl)-4-nitrobenzamide
3-nitro-N-({5-[(4-{3-[(trifluoromethyl)sulfanyl]anilino}piperidin-1-yl)sulfonyl]thien-2-yl}methyl)benzamide
4-nitro-N-({5-[(4-{3-[(trifluoromethyl)sulfanyl]anilino}piperidin-1-yl)sulfonyl]thien-2-yl}methyl)benzamide
3-nitro-N-[(5-{[4-({3-nitropyridin-2-yl}amino)piperidin-1-yl]sulfonyl}thien-2-yl)methyl]benzamide
N-{[5-({4-[(2,2-dioxido-1,3-dihydro-2-benzothien-5-yl)amino]piperidin-1-yl}sulfonyl)thien-2-yl]methyl}-3-nitrobenzamide
N-[(5-{[4-(2,3-dihydro-1H-inden-5-ylamino)piperidin-1-yl]sulfonyl}thien-2-yl)methyl]-3-nitrobenzamide
3-nitro-N-[(5-{[4-(2-propylanilino)piperidin-1-yl]sulfonyl}thien-2-yl)methyl]benzamide
3-nitro-N-[(5-{[4-(4-propylanilino)piperidin-1-yl]sulfonyl}thien-2-yl)methyl]benzamide
N-[(5-{[4-(3-tert-butylanilino)piperidin-1-yl]sulfonyl}thien-2-yl)methyl]-3-nitrobenzamide
3-nitro-N-{[5-({4-[3-(1,3-oxazol-5-yl)anilino]piperidin-1-yl}sulfonyl)thien-2-yl]methyl}benzamide
3-nitro-N-[(5-{[4-(2-phenylethyl)piperidin-1-yl]sulfonyl}thien-2-yl)methyl]benzamide
N-({5-[(4-{[3-chloro-5-(trifluoromethyl)pyridin-2-yl]amino}piperidin-1-yl)sulfonyl]thien-2-yl}methyl)-3-nitrobenzamide
N-[(5-{[4-([1,1′-biphenyl]-3-ylamino)piperidin-1-yl]sulfonyl}thien-2-yl)methyl]-3-nitrobenzamide
N-[(5-{[4-(3-benzylanilino)piperidin-1-yl]sulfonyl}thien-2-yl)methyl]-3-nitrobenzamide
3-nitro-N-{[5-({4-[3-(morpholin-4-ylsulfonyl)anilino]piperidin-1-yl}sulfonyl)thien-2-yl]methyl}benzamide
3-nitro-N-[(5-{[4-(3-propylphenoxy)piperidin-1-yl]sulfonyl}thien-2-yl)methyl]benzamide
4-nitro-N-[(5-{[4-(pyrimidin-2-ylamino)piperidin-1-yl]sulfonyl}thien-2-yl)methyl]benzamide
N-{[5-({4-[(3-aminopyridin-2-yl)amino]piperidin-1-yl}sulfonyl)thien-2-yl]methyl}-4-nitrobenzamide
4-nitro-N-[(5-{[4-({3-nitropyridin-2-yl}amino)piperidin-1-yl]sulfonyl}thien-2-yl)methyl]benzamide
N-[(5-{[4-(2,3-dihydro-1H-inden-5-ylamino)piperidin-1-yl]sulfonyl}thien-2-yl)methyl]-4-nitrobenzamide
4-nitro-N-[(5-{[4-(2-propylanilino)piperidin-1-yl]sulfonyl}thien-2-yl)methyl]benzamide
4-nitro-N-[(5-{[4-(4-propylanilino)piperidin-1-yl]sulfonyl}thien-2-yl)methyl]benzamide
N-[(5-{[4-(3-tert-butylanilino)piperidin-1-yl]sulfonyl}thien-2-yl)methyl]-4-nitrobenzamide
4-nitro-N-{[5-({4-[3-(1,3-oxazol-5-yl)anilino]piperidin-1-yl}sulfonyl)thien-2-yl]methyl}benzamide
4-nitro-N-[(5-{[4-(2-phenylethyl)piperidin-1-yl]sulfonyl}thien-2-yl)methyl]benzamide
N-({5-[(4-{[3-chloro-5-(trifluoromethyl)pyridin-2-yl]amino}piperidin-1-yl)sulfonyl]thien-2-yl}methyl)-4-nitrobenzamide
N-[(5-{[4-([1,1′-biphenyl]-3-ylamino)piperidin-1-yl]sulfonyl}thien-2-yl)methyl]-4-nitrobenzamide
N-[(5-{[4-(3-benzylanilino)piperidin-1-yl]sulfonyl}thien-2-yl)methyl]-4-nitrobenzamide
4-nitro-N-{[5-({4-[3-(morpholin-4-ylsulfonyl)anilino]piperidin-1-yl}sulfonyl)thien-2-yl]methyl}benzamide
N-[(5-{[4-(2-aminoanilino)piperidin-1-yl]sulfonyl}thien-2-yl)methyl]-3-nitrobenzamide
3-nitro-N-[(5-{[4-(pyrimidin-2-ylamino)piperidin-1-yl]sulfonyl}thien-2-yl)methyl]benzamide
N-{[5-({4-[(3-aminopyridin-2-yl)amino]piperidin-1-yl}sulfonyl)thien-2-yl]methyl}-3-nitrobenzamide
N-({5-[(4-{2-nitro-4-[(trifluoromethyl)sulfonyl]anilino}piperidin-1-yl)sulfonyl]thien-2-yl}methyl)-3-methoxybenzamide
3-nitro-N-[(5-{[4-(3-phenylpropyl)piperazin-1-yl]sulfonyl}thien-2-yl)methyl]benzamide
3-nitro-N-({5-[(4-{[4-(trifluoromethyl)pyrimidin-2-yl]amino}piperidin-1-yl)sulfonyl]thien-2-yl}methyl)benzamide
N-[(5-{[4-(3-cyclohexyl-4-hydroxyanilino)piperidin-1-yl]sulfonyl}thien-2-yl)methyl]-3-nitrobenzamide
N-({5-[(4-{3-[(butylamino)sulfonyl]anilino}piperidin-1-yl)sulfonyl]thien-2-yl}methyl)-3-nitrobenzamide
N-[(5-{[4-(3-ethylanilino)piperidin-1-yl]sulfonyl}thien-2-yl)methyl]-3-nitrobenzamide
3-nitro-N-[(5-{[4-(5,6,7,8-tetrahydronaphthalen-1-ylamino)piperidin-1-yl]sulfonyl}thien-2-yl)methyl]benzamide
4-nitro-N-[(5-{[4-(3-propylphenoxy)piperidin-1-yl]sulfonyl}thien-2-yl)methyl]benzamide
N-[(5-{[4-(2,4-difluorobenzoyl)piperidin-1-yl]sulfonyl}thien-2-yl)methyl]-3-nitrobenzamide
N-[(5-{[4-(2,4-difluorobenzoyl)piperidin-1-yl]sulfonyl}thien-2-yl)methyl]-3-methoxybenzamide
2-Hydroxy-N-({5-[(4-{3-[(trifluoromethyl)sulfonyl]anilino}piperidin-1-yl)sulfonyl]thien-2-yl}methyl)benzamide
N-[(5-{[4-(1H-1,2,3-benzotriazol-1-yl)piperidin-1-yl]sulfonyl}thien-2-yl)methyl]-3-methoxybenzamide
N-[(5-{[4-(1H-1,2,3-benzotriazol-1-yl)piperidin-1-yl]sulfonyl}thien-2-yl)methyl]-2-hydroxybenzamide
N-{[5-({4-[4-(1,3-dithiolan-2-yl)anilino]piperidin-1-yl}sulfonyl)thien-2-yl]methyl}-3-nitrobenzamide
3-methoxy-N-[(5-{[4-(3-methoxyanilino)piperidin-1-yl]sulfonyl}thien-2-yl)methyl]benzamide
3-methoxy-N-{[5-({4-[3-(trifluoromethyl)anilino]piperidin-1-yl}sulfonyl)thien-2-yl]methyl}benzamide
N-{[5-({4-[3-(dimethylamino)anilino]piperidin-1-yl}sulfonyl)thien-2-yl]methyl}-3-methoxybenzamide
3-methoxy-N-[(5-{[4-(3-propylanilino)piperidin-1-yl]sulfonyl}thien-2-yl)methyl]benzamide
3-methoxy-N-{[5-({4-[3-(methylsulfonyl)anilino]piperidin-1-yl}sulfonyl)thien-2-yl]methyl}benzamide
3-methoxy-N-{[5-({4-[3-(methylsulfanyl)anilino]piperidin-1-yl}sulfonyl)thien-2-yl]methyl}benzamide
N-{[5-({4-[3-(aminosulfonyl)anilino]piperidin-1-yl}sulfonyl)thien-2-yl]methyl}-3-methoxybenzamide
methyl 3-({1-[(5-{[(3-methoxybenzoyl)amino]-methyl}thien-2-yl)sulfonyl]-piperidin-4-yl}amino)-benzoate
N-{[5-({4-[3-(aminocarbonyl)anilino]piperidin-1-yl}sulfonyl)thien-2-yl]methyl}-3-methoxybenzamide
3-methoxy-N-[(5-{[4-(2-methoxyanilino)piperidin-1-yl]sulfonyl}thien-2-yl)methyl]benzamide
N-({5-[(4-{3-nitroanilino}piperidin-1-yl)sulfonyl]thien-2-yl}methyl)-3-methoxybenzamide
3-methoxy-N-{[5-({4-[2-(trifluoromethyl)anilino]piperidin-1-yl}sulfonyl)thien-2-yl]methyl}benzamide
N-({5-[(4-{2-nitroanilino}piperidin-1-yl)sulfonyl]thien-2-yl}methyl)-3-methoxybenzamide
N-{[5-({4-[4-(aminocarbonyl)anilino]piperidin-1-yl}sulfonyl)thien-2-yl]methyl}-3-methoxybenzamide
N-{[5-({4-[4-(1,3-dithiolan-2-yl)anilino]piperidin-1-yl}sulfonyl)thien-2-yl]methyl}-3-methoxybenzamide
N-[(5-{[4-(3-chloroanilino)piperidin-1-yl]sulfonyl}thien-2-yl)methyl]-3-methoxybenzamide
N-[(5-{[4-(4-chloroanilino)piperidin-1-yl]sulfonyl}thien-2-yl)methyl]-3-methoxybenzamide
3-methoxy-N-({5-[(4-{4-[(trifluoromethyl)sulfonyl]anilino}piperidin-1-yl)sulfonyl]thien-2-yl}methyl)benzamide
N-({5-[(4-{3-[amino(imino)methyl]anilino}piperidin-1-yl)sulfonyl]thien-2-yl}methyl)-3-methoxybenzamide
N-({5-[(4-{3-[(2-hydroxyethyl)sulfonyl]anilino}piperidin-1-yl)sulfonyl]thien-2-yl}methyl)-3-methoxybenzamide
3-methoxy-N-({5-[(4-{3-[(trifluoromethyl)sulfonyl]anilino}piperidin-1-yl)sulfonyl]thien-2-yl}methyl)benzamide
N-({5-[(4-anilinopiperidin-1-yl)sulfonyl]thien-2-yl}methyl)-3-methoxybenzamide
3-methoxy-N-({5-[(4-{3-[(trifluoromethyl)sulfanyl]anilino}piperidin-1-yl)sulfonyl]thien-2-yl}methyl)benzamide
N-[(5-{[4-(4-hydroxyanilino)piperidin-1-yl]sulfonyl}thien-2-yl)methyl]-3-methoxybenzamide
3-nitro-N-({5-[(4-{3-[(trifluoromethyl)sulfanyl]anilino}piperidin-1-yl)sulfonyl]thien-2-yl}methyl)benzamide
4-nitro-N-({5-[(4-{3-[(trifluoromethyl)sulfanyl]anilino}piperidin-1-yl)sulfonyl]thien-2-yl}methyl)benzamide
N-[(5-{[4-(2-hydroxyanilino)piperidin-1-yl]sulfonyl}thien-2-yl)methyl]-3-methoxybenzamide
3-methoxy-N-[(5-{[4-(pyrimidin-2-ylamino)piperidin-1-yl]sulfonyl}thien-2-yl)methyl]benzamide
N-{[5-({4-[(3-aminopyridin-2-yl)amino]piperidin-1-yl}sulfonyl)thien-2-yl]methyl}-3-methoxybenzamide
N-[(5-{[4-({3-nitropyridin-2-yl}amino)piperidin-1-yl]sulfonyl}thien-2-yl)methyl]-3-methoxybenzamide
N-{[5-({4-[(2,2-dioxido-1,3-dihydro-2-benzothien-5-yl)amino]piperidin-1-yl}sulfonyl)thien-2-yl]methyl}-3-methoxybenzamide
N-[(5-{[4-(2,3-dihydro-1H-inden-5-ylamino)piperidin-1-yl]sulfonyl}thien-2-yl)methyl]-3-methoxybenzamide
3-methoxy-N-[(5-{[4-(2-propylanilino)piperidin-1-yl]sulfonyl}thien-2-yl)methyl]benzamide
3-methoxy-N-[(5-{[4-(4-propylanilino)piperidin-1-yl]sulfonyl}thien-2-yl)methyl]benzamide
N-[(5-{[4-(3-tert-butylanilino)piperidin-1-yl]sulfonyl}thien-2-yl)methyl]-3-methoxybenzamide
N-({5-[(4-{[3-chloro-5-(trifluoromethyl)pyridin-2-yl]amino}piperidin-1-yl)sulfonyl]thien-2-yl}methyl)-3-methoxybenzamide
3-methoxy-N-{[5-({4-[3-(1,3-oxazol-5-yl)anilino]piperidin-1-yl}sulfonyl)thien-2-yl]methyl}benzamide
N-[(5-{[4-([1,1′-biphenyl]-3-ylamino)piperidin-1-yl]sulfonyl}thien-2-yl)methyl]-3-methoxybenzamide
3-methoxy-N-[(5-{[4-(3-propylphenoxy)piperidin-1-yl]sulfonyl}thien-2-yl)methyl]benzamide
3-methoxy-N-{[5-({4-[3-(morpholin-4-ylsulfonyl)anilino]piperidin-1-yl}sulfonyl)thien-2-yl]methyl}benzamide
3-methoxy-N-[(5-{[4-(2-phenylethyl)piperidin-1-yl]sulfonyl}thien-2-yl)methyl]benzamide
N-[(5-{[4-(3-benzylanilino)piperidin-1-yl]sulfonyl}thien-2-yl)methyl]-3-methoxybenzamide
3-methoxy-N-[(5-{[4-(3-phenylpropyl)piperazin-1-yl]sulfonyl}thien-2-yl)methyl]benzamide
3-methoxy-N-({5-[(4-{[4-(trifluoromethyl)pyrimidin-2-yl]amino}piperidin-1-yl)sulfonyl]thien-2-yl}methyl)benzamide
N-[(5-{[4-(3-cyclohexyl-4-hydroxyanilino)piperidin-1-yl]sulfonyl}thien-2-yl)methyl]-3-methoxybenzamide
N-({5-[(4-{3-[(butylamino)sulfonyl]anilino}piperidin-1-yl)sulfonyl]thien-2-yl}methyl)-3-methoxybenzamide
N-[(5-{[4-(3-ethylanilino)piperidin-1-yl]sulfonyl}thien-2-yl)methyl]-3-methoxybenzamide
3-methoxy-N-[(5-{[4-(5,6,7,8-tetrahydronaphthalen-1-ylamino)piperidin-1-yl]sulfonyl}thien-2-yl)methyl]benzamide
N-[(5-{[4-(1H-1,2,3-benzotriazol-1-yl)piperidin-1-yl]sulfonyl}thien-2-yl)methyl]-5-nitro-1H-pyrazole-3-carboxamide
N-[(5-{[4-(1H-1,2,3-benzotriazol-1-yl)piperidin-1-yl]sulfonyl}thien-2-yl)methyl]-2-oxo-1,2-dihydropyridine-3-carboxamide
N-[(5-{[4-(1H-1,2,3-benzotriazol-1-yl)piperidin-1-yl]sulfonyl}thien-2-yl)methyl]-2-thioxo-1,2-dihydropyridine-3-carboxamide
N-[(5-{[4-(1H-1,2,3-benzotriazol-1-yl)piperidin-1-yl]sulfonyl}thien-2-yl)methyl]-3,4-dihydroxybenzamide
N-[(5-{[4-(1H-1,2,3-benzotriazol-1-yl)piperidin-1-yl]sulfonyl}thien-2-yl)methyl]pyridine-2-carboxamide
N-[(5-{[4-(hexyloxy)piperidin-1-yl]sulfonyl}thien-2-yl)methyl]-3-methoxybenzamide
N-({5-[(4-heptanoylpiperidin-1-yl)sulfonyl]thien-2-yl}methyl)-3-methoxybenzamide
4-chloro-N-[(5-{[4-(3-propylanilino)piperidin-1-yl]sulfonyl}-2-furyl)methyl]benzamide
4-chloro-N-[(5-{[4-(3-chloroanilino)piperidin-1-yl]sulfonyl}-2-furyl)methyl]benzamide
4-chloro-N-[(5-{[4-(3-methoxyanilino)piperidin-1-yl]sulfonyl}-2-furyl)methyl]benzamide
4-chloro-N-{[5-({4-[3-(trifluoromethyl)anilino]piperidin-1-yl}sulfonyl)-2-furyl]methyl}benzamide
4-chloro-N-{[5-({4-[3-(dimethylamino)anilino]piperidin-1-yl}sulfonyl)-2-furyl]methyl}benzamide
4-chloro-N-{[5-({4-[3-(methylsulfonyl)anilino]piperidin-1-yl}sulfonyl)-2-furyl]methyl}benzamide
4-chloro-N-{[5-({4-[3-(methylsulfanyl)anilino]piperidin-1-yl}sulfonyl)-2-furyl]methyl}benzamide
N-{[5-({4-[3-(aminosulfonyl)anilino]piperidin-1-yl}sulfonyl)-2-furyl]methyl}-4-chlorobenzamide
methyl 3-({1-[(5-{[(4-chlorobenzoyl)amino]methyl}-2-furyl)sulfonyl]piperidin-4-yl}amino)benzoate
3-({1-[(5-{[(4-chlorobenzoyl)amino]methyl}-2-furyl)sulfonyl]piperidin-4-yl}amino)benzamide
4-chloro-N-({5-[(4-{3-nitroanilino}piperidin-1-yl)sulfonyl]-2-furyl}methyl)benzamide
4-chloro-N-[(5-{[4-(2-methoxyanilino)piperidin-1-yl]sulfonyl}-2-furyl)methyl]benzamide
4-chloro-N-{[5-({4-[2-(trifluoromethyl)anilino]piperidin-1-yl}sulfonyl)-2-furyl]methyl}benzamide
4-chloro-N-({5-[(4-{2-nitroanilino}piperidin-1-yl)sulfonyl]-2-furyl}methyl)benzamide
4-chloro-N-[(5-{[4-(4-chloroanilino)piperidin-1-yl]sulfonyl}-2-furyl)methyl]benzamide
4-chloro-N-{[5-({4-[4-(trifluoromethyl)anilino]piperidin-1-yl}sulfonyl)-2-furyl]methyl}benzamide
4-chloro-N-({5-[(4-{4-[(trifluoromethyl)sulfonyl]anilino}piperidin-1-yl)sulfonyl]-2-furyl}methyl)benzamide
N-{[5-({4-[4-(aminocarbonyl)anilino]piperidin-1-yl}sulfonyl)-2-furyl]methyl}-4-chlorobenzamide
4-chloro-N-{[5-({4-[4-(1,3-dithiolan-2-yl)anilino]piperidin-1-yl}sulfonyl)-2-furyl]methyl}benzamide
N-({5-[(4-{3-[amino(imino)methyl]anilino}piperidin-1-yl)sulfonyl]-2-furyl}methyl)-4-chlorobenzamide
4-chloro-N-({5-[(4-{3-[(trifluoromethyl)sulfonyl]anilino}piperidin-1-yl)sulfonyl]-2-furyl}methyl)benzamide
N-({5-[(4-anilinopiperidin-1-yl)sulfonyl]-2-furyl}methyl)-4-chlorobenzamide
4-nitro-N-({5-[(4-{3-[(trifluoromethyl)sulfanyl]anilino}piperidin-1-yl)sulfonyl]2-furyl}methyl)benzamide
4-chloro-N-({5-[(3-{3-[(trifluoromethyl)sulfonyl]anilino}pyrrolidin-1-yl)sulfonyl]thien-2-yl}methyl)benzamide
4-chloro-N-({5-[(4-{3-[(trifluoromethyl)sulfonyl]anilino}azepan-1-yl)sulfonyl]thien-2-yl}methyl)benzamide
5-{[(3-methoxybenzoyl)amino]methyl}-2-[(4-{3-[(trifluoromethyl)sulfonyl]-anilino}piperidin-1-yl)sulfonyl]thiophene-3-carboxylic acid
5-{[(3-methoxybenzoyl)amino]methyl}-2-{[4-(octylamino)piperidin-1-yl]sulfonyl}thiophene-3-carboxylic acid
N-(2-hydroxyethyl)-5-{[(3-methoxybenzoyl)amino]methyl}-2-[(4-{3-[(trifluoromethyl)sulfonyl]anilino}piperidin-1-yl)sulfonyl]thiophene-3-carboxamide
N-({4-(hydrazinocarbonyl)-5-[(4-{3-[(trifluoromethyl)sulfonyl]anilino}-piperidin-1-yl)sulfonyl]thien-2-yl}methyl)-3-methoxybenzamide
5-{[(3-methoxybenzoyl)amino]methyl}-2-[(4-{3-[(trifluoromethyl)sulfonyl]-anilino}piperidin-1-yl)sulfonyl]thiophene-3-carboxamide
N-[2-(dimethylamino)ethyl]-5-{[(3-methoxybenzoyl)amino]methyl}-2-[(4-{3-[(trifluoromethyl)sulfonyl]anilino}piperidin-1-yl)sulfonyl]thiophene-3-carboxamide
N-({4-(hydroxymethyl)-5-[(4-{3-[(trifluoromethyl)sulfonyl]anilino}piperidin-1-yl)sulfonyl]thien-2-yl}methyl)-3-methoxybenzamide
4-chloro-N-[(5-{[4-(hexylamino)piperidin-1-yl]sulfonyl}thien-2-yl)methyl]benzamide
3-Methoxy-N-{[5-({4-[(4-trifluoromethylbenzyl)amino]piperidin-1-yl}sulfonyl)thien-2-yl]methyl}benzamide
4-chloro-N-[(5-{[4-(1,3-thiazol-2-ylamino)piperidin-1-yl]sulfonyl}thien-2-yl)methyl]benzamide
4-chloro-N-[(5-{[4-(heptylamino)piperidin-1-yl]sulfonyl}thien-2-yl)methyl]benzamide
4-chloro-N-[(5-{[4-(pentylamino)piperidin-1-yl]sulfonyl}thien-2-yl)methyl]benzamide
4-chloro-N-[(5-{[4-(butylamino)piperidin-1-yl]sulfonyl}thien-2-yl)methyl]benzamide
4-chloro-N-[(5-{[4-(dodecylamino)piperidin-1-yl]sulfonyl}thien-2-yl)methyl]benzamide
4-chloro-N-{[5-({4-[(2-cyclohexylethyl)amino]piperidin-1-yl}sulfonyl)thien-2-yl]methyl}benzamide
4-chloro-N-{[5-({4-[(cyclohexylmethyl)amino]piperidin-1-yl}sulfonyl)thien-2-yl]methyl}benzamide
4-chloro-N-({5-[(4-{[(1R)-1-cyclohexylethyl]amino}piperidin-1-yl)sulfonyl]thien-2-yl}methyl)benzamide
N-{[5-({4-[(1R,2R,4S)-bicyclo[2.2.1]hept-2-ylamino]piperidin-1-yl}sulfonyl)thien-2-yl]methyl}-4-chlorobenzamide
4-chloro-N-{[5-({4-[(2-propoxyethyl)amino]piperidin-1-yl}sulfonyl)thien-2-yl]methyl}benzamide
N-{[5-({4-[(1-adamantylmethyl)amino]piperidin-1-yl}sulfonyl)thien-2-yl]methyl}-4-chlorobenzamide
4-chloro-N-{[5-({4-[(2-pyridin-2-ylethyl)amino]piperidin-1-yl}sulfonyl)thien-2-yl]methyl}benzamide
4-chloro-N-{[5-({4-[(2-piperidin-1-ylethyl)amino]piperidin-1-yl}sulfonyl)thien-2-yl]methyl}benzamide
4-chloro-N-{[5-({4-[(2-ethylhexyl)amino]piperidin-1-yl}sulfonyl)thien-2-yl]methyl}benzamide
4-chloro-N-({5-[(4-{[3-(1H-imidazol-1-yl)propyl]amino}piperidin-1-yl)sulfonyl]thien-2-yl}methyl)benzamide
4-chloro-N-[(5-{[4-(octylamino)piperidin-1-yl]sulfonyl}thien-2-yl)methyl]benzamide
N-[(5-{[4-(heptylamino)piperidin-1-yl]sulfonyl}thien-2-yl)methyl]-3-methoxybenzamide
3-methoxy-N-[(5-{[4-(octylamino)piperidin-1-yl]sulfonyl}thien-2-yl)methyl]benzamide
3-methoxy-N-[(5-{[4-(pentylamino)piperidin-1-yl]sulfonyl}thien-2-yl)methyl]benzamide
N-[(5-{[4-(butylamino)piperidin-1-yl]sulfonyl}thien-2-yl)methyl]-3-methoxybenzamide
N-[(5-{[4-(dodecylamino)piperidin-1-yl]sulfonyl}thien-2-yl)methyl]-3-methoxybenzamide
N-{[5-({4-[(2-cyclohexylethyl)amino]piperidin-1-yl}sulfonyl)thien-2-yl]methyl}-3-methoxybenzamide
N-({5-[(4-{[(1R)-1-cyclohexylethyl]amino}piperidin-1-yl)sulfonyl]thien-2-yl}methyl)-3-methoxybenzamide
N-{[5-({4-[(1R,2R,4S)-bicyclo[2.2.1]hept-2-ylamino]piperidin-1-yl}sulfonyl)thien-2-yl]methyl}-3-methoxybenzamide
3-methoxy-N-{[5-({4-[(2-propoxyethyl)amino]piperidin-1-yl}sulfonyl)thien-2-yl]methyl}benzamide
N-{[5-({4-[(1-adamantylmethyl)amino]piperidin-1-yl}sulfonyl)thien-2-yl]methyl}-3-methoxybenzamide
N-{[5-({4-[(3,3-diethoxypropyl)amino]piperidin-1-yl}sulfonyl)thien-2-yl]methyl}-3-methoxybenzamide
3-methoxy-N-{[5-({4-[(3-morpholin-4-ylpropyl)amino]piperidin-1-yl}sulfonyl)thien-2-yl]methyl}benzamide
3-methoxy-N-{[5-({4-[(2-pyridin-2-ylethyl)amino]piperidin-1-yl}sulfonyl)thien-2-yl]methyl}benzamide
3-methoxy-N-{[5-({4-[(2-piperidin-1-ylethyl)amino]piperidin-1-yl}sulfonyl)thien-2-yl]methyl}benzamide
N-{[5-({4-[(2-ethylhexyl)amino]piperidin-1-yl}sulfonyl)thien-2-yl]methyl}-3-methoxybenzamide
N-({5-[(4-{[3-(1H-imidazol-1-yl)propyl]amino}piperidin-1-yl)sulfonyl]thien-2-yl}methyl)-3-methoxybenzamide
N-[(5-{[4-(hexylamino)piperidin-1-yl]sulfonyl}thien-2-yl)methyl]-3-methoxybenzamide
N-[(5-{[4-(heptylamino)azepan-1-yl]sulfonyl}thien-2-yl)methyl]-3-methoxybenzamide
3-methoxy-N-[(5-{[4-(octylamino)azepan-1-yl]sulfonyl}thien-2-yl)methyl]benzamide
3-methoxy-N-[(5-{[4-(pentylamino)azepan-1-yl]sulfonyl}thien-2-yl)methyl]benzamide
N-[(5-{[4-(butylamino)azepan-1-yl]sulfonyl}thien-2-yl)methyl]-3-methoxybenzamide
N-[(5-{[4-(dodecylamino)azepan-1-yl]sulfonyl}thien-2-yl)methyl]-3-methoxybenzamide
N-{[5-({4-[(2-cyclohexylethyl)amino]azepan-1-yl}sulfonyl)thien-2-yl]methyl}-3-methoxybenzamide
N-({5-[(4-{[(1R)-1-cyclohexylethyl]amino}azepan-1-yl)sulfonyl]thien-2-yl}methyl)-3-methoxybenzamide
N-{[5-({4-[(1R,2R,4S)-bicyclo[2.2.1]hept-2-ylamino]azepan-1-yl}sulfonyl)thien-2-yl]methyl}-3-methoxybenzamide
3-methoxy-N-{[5-({4-[(2-propoxyethyl)amino]azepan-1-yl}sulfonyl)thien-2-yl]methyl}benzamide
N-{[5-({4-[(cyclohexylmethyl)amino]azepan-1-yl}sulfonyl)thien-2-yl]methyl}-3-methoxybenzamide
N-{[5-({4-[(1-adamantylmethyl)amino]azepan-1-yl}sulfonyl)thien-2-yl]methyl}-3-methoxybenzamide
3-methoxy-N-{[5-({4-[(3-morpholin-4-ylpropyl)amino]azepan-1-yl}sulfonyl)thien-2-yl]methyl}benzamide
3-methoxy-N-{[5-({4-[(2-pyridin-2-ylethyl)amino]azepan-1-yl}sulfonyl)thien-2-yl]methyl}benzamide
3-methoxy-N-{[5-({4-[(2-piperidin-1-ylethyl)amino]azepan-1-yl}sulfonyl)thien-2-yl]methyl}benzamide
N-{[5-({4-[(2-ethylhexyl)amino]azepan-1-yl}sulfonyl)thien-2-yl]methyl}-3-methoxybenzamide
N-({5-[(4-{[3-(1H-imidazol-1-yl)propyl]amino}azepan-1-yl)sulfonyl]thien-2-yl}methyl)-3-methoxybenzamide
4-chloro-N-[(5-{[4-(heptylamino)azepan-1-yl]sulfonyl}thien-2-yl)methyl]benzamide
4-chloro-N-[(5-{[4-(octylamino)azepan-1-yl]sulfonyl}thien-2-yl)methyl]benzamide
4-chloro-N-[(5-{[4-(pentylamino)azepan-1-yl]sulfonyl}thien-2-yl)methyl]benzamide
N-[(5-{[4-(butylamino)azepan-1-yl]sulfonyl}thien-2-yl)methyl]-4-chlorobenzamide
4-chloro-N-[(5-{[4-(dodecylamino)azepan-1-yl]sulfonyl}thien-2-yl)methyl]benzamide
4-chloro-N-{[5-({4-[(2-cyclohexylethyl)amino]azepan-1-yl}sulfonyl)thien-2-yl]methyl}benzamide
N-{[5-({4-[(1R,2R,4S)-bicyclo[2.2.1]hept-2-ylamino]azepan-1-yl}sulfonyl)thien-2-yl]methyl}-4-chlorobenzamide
4-chloro-N-{[5-({4-[(2-propoxyethyl)amino]azepan-1-yl}sulfonyl)thien-2-yl]methyl}benzamide
4-chloro-N-{[5-({4-[(2-ethylhexyl)amino]azepan-1-yl}sulfonyl)thien-2-yl]methyl}benzamide
4-chloro-N-[(5-{[4-(hexylamino)azepan-1-yl]sulfonyl}thien-2-yl)methyl]benzamide
N-[(5-{[4-(hexylamino)azepan-1-yl]sulfonyl}thien-2-yl)methyl]-3-methoxybenzamide
3-methoxy-N-[(5-{[4-({2-[3-(trifluoromethyl)phenyl]ethyl}amino)piperidin-1-yl]sulfonyl}thien-2-yl)methyl]benzamide
3-methoxy-N-({5-[(4-{[2-(4-methylphenyl)ethyl]amino}piperidin-1-yl)sulfonyl]thien-2-yl}methyl)benzamide
3-methoxy-N-({5-[(4-{[(1S,2R)-2-phenylcyclopropyl]amino}piperidin-1-yl)sulfonyl]thien-2-yl}methyl)benzamide
3-methoxy-N-{[5-({4-[(1-naphthylmethyl)amino]piperidin-1-yl}sulfonyl)thien-2-yl]methyl}benzamide
3-methoxy-N-{[5-({4-[(2-phenylpropyl)amino]piperidin-1-yl}sulfonyl)thien-2-yl]methyl}benzamide
N-({5-[(4-{[2-(4-hydroxyphenyl)ethyl]amino}piperidin-1-yl)sulfonyl]thien-2-yl}methyl)-3-methoxybenzamide
3-methoxy-N-{[5-({4-[(3-phenylpropyl)amino]piperidin-1-yl}sulfonyl)thien-2-yl]methyl}benzamide
N-{[5-({4-[(2,3-dihydroxypropyl)amino]piperidin-1-yl}sulfonyl)thien-2-yl]methyl}-3-methoxybenzamide
N-{[5-({4-[(2-hydroxyethyl)amino]piperidin-1-yl}sulfonyl)thien-2-yl]methyl}-3-methoxybenzamide
3-methoxy-N-[(5-{[4-(nonylamino)piperidin-1-yl]sulfonyl}thien-2-yl)methyl]benzamide
3-methoxy-N-[(5-{[4-(decylamino)piperidin-1-yl]sulfonyl}thien-2-yl)methyl]benzamide
3-methoxy-N-[(5-{[4-(ethylamino)piperidin-1-yl]sulfonyl}thien-2-yl)methyl]benzamide
N-{[5-({4-[(2-[1,1′-biphenyl]-4-ylethyl)amino]piperidin-1-yl}sulfonyl)thien-2-yl]methyl}-3-methoxybenzamide
N-{[5-({4-[([1,1′-biphenyl]-3-ylmethyl)amino]piperidin-1-yl}sulfonyl)thien-2-yl]methyl}-3-methoxybenzamide
3-methoxy-N-{[5-({4-[(2-thien-2-ylethyl)amino]piperidin-1-yl}sulfonyl)thien-2-yl]methyl}benzamide
3-methoxy-N-[(5-{[4-({4-[(trifluoromethyl)sulfonyl]benzyl}amino)piperidin-1-yl]sulfonyl}thien-2-yl)methyl]benzamide
3-methoxy-N-{[5-({4-[(quinolin-4-ylmethyl)amino]piperidin-1-yl}sulfonyl)thien-2-yl]methyl}benzamide
N-{[5-({4-[([1,1′-biphenyl]-4-ylmethyl)amino]-1-piperidinyl}sulfonyl)-2-thienyl]methyl}-3-methoxybenzamide
4-chloro-N-{[5-({4-[(2-{[(trifluoromethyl)sulfonyl]amino}ethyl)amino]-1-piperidinyl}sulfonyl)-2-thienyl]methyl}benzamide
4-chloro-N-[(5-{[4-(propylamino)-1-piperidinyl]sulfonyl}-2-thienyl)methyl]benzamide
4-chloro-N-[(5-{[4-({4-[(trifluoromethyl)sulfonyl]benzyl}amino)-1-piperidinyl]sulfonyl}-2-thienyl)methyl]benzamide
4-chloro-N-{[5-({4-[(3,4-dihydroxybenzyl)amino]-1-piperidinyl}sulfonyl)-2-thienyl]methyl}benzamide
methyl [{1-[(5-{[(4-chlorobenzoyl)amino]methyl}-2-thienyl)sulfonyl]-4-piperidinyl}(hexyl)amino]acetate
tert-butyl [{1-[(5-{[(4-chlorobenzoyl)amino]methyl}-2-thienyl)sulfonyl]-4-piperidinyl}(hexyl)amino]acetate
[{1-[(5-{[(4-chlorobenzoyl)amino]methyl}-2-thienyl)sulfonyl]-4-piperidinyl}(hexyl)amino]acetic acid
N-[(5-{[3-(heptylamino)pyrrolidin-1-yl]sulfonyl}thien-2-yl)methyl]-3-methoxybenzamide
3-methoxy-N-[(5-{[3-(octylamino)pyrrolidin-1-yl]sulfonyl}thien-2-yl)methyl]benzamide
3-methoxy-N-[(5-{[3-(pentylamino)pyrrolidin-1-yl]sulfonyl}thien-2-yl)methyl]benzamide
N-[(5-{[3-(butylamino)pyrrolidin-1-yl]sulfonyl}thien-2-yl)methyl]-3-methoxybenzamide
N-[(5-{[3-(dodecylamino)pyrrolidin-1-yl]sulfonyl}thien-2-yl)methyl]-3-methoxybenzamide
N-{[5-({3-[(2-cyclohexylethyl)amino]pyrrolidin-1-yl}sulfonyl)thien-2-yl]methyl}-3-methoxybenzamide
N-({5-[(3-{[(1R)-1-cyclohexylethyl]amino}pyrrolidin-1-yl)sulfonyl]thien-2-yl}methyl)-3-methoxybenzamide
N-{[5-({3-[(1R,2R,4S)-bicyclo[2.2.1]hept-2-ylamino]pyrrolidin-1-yl}sulfonyl)thien-2-yl]methyl}-3-methoxybenzamide
3-methoxy-N-{[5-({3-[(2-propoxyethyl)amino]pyrrolidin-1-yl}sulfonyl)thien-2-yl]methyl}benzamide
N-{[5-({3-[(cyclohexylmethyl)amino]pyrrolidin-1-yl}sulfonyl)thien-2-yl]methyl}-3-methoxybenzamide
N-{[5-({3-[(1-adamantylmethyl)amino]pyrrolidin-1-yl}sulfonyl)thien-2-yl]methyl}-3-methoxybenzamide
3-methoxy-N-{[5-({3-[(3-morpholin-4-ylpropyl)amino]pyrrolidin-1-yl}sulfonyl)thien-2-yl]methyl}benzamide
3-methoxy-N-{[5-({3-[(2-pyridin-2-ylethyl)amino]pyrrolidin-1-yl}sulfonyl)thien-2-yl]methyl}benzamide
3-methoxy-N-{[5-({3-[(2-piperidin-1-ylethyl)amino]pyrrolidin-1-yl}sulfonyl)thien-2-yl]methyl}benzamide
N-{[5-({3-[(2-ethylhexyl)amino]pyrrolidin-1-yl}sulfonyl)thien-2-yl]methyl}-3-methoxybenzamide
N-[(5-{[3-(hexylamino)pyrrolidin-1-yl]sulfonyl}thien-2-yl)methyl]-3-methoxybenzamide
4-chloro-N-[(5-{[3-(heptylamino)pyrrolidin-1-yl]sulfonyl}thien-2-yl)methyl]benzamide
4-chloro-N-[(5-{[3-(hexylamino)pyrrolidin-1-yl]sulfonyl}thien-2-yl)methyl]benzamide
4-chloro-N-[(5-{[3-(pentylamino)pyrrolidin-1-yl]sulfonyl}thien-2-yl)methyl]benzamide
N-[(5-{[3-(butylamino)pyrrolidin-1-yl]sulfonyl}thien-2-yl)methyl]-4-chlorobenzamide
4-chloro-N-{[5-({3-[(2-cyclohexylethyl)amino]pyrrolidin-1-yl}sulfonyl)thien-2-yl]methyl}benzamide
N-{[5-({3-[(1R,2R,4S)-bicyclo[2.2.1]hept-2-ylamino]pyrrolidin-1-yl}sulfonyl)thien-2-yl]methyl}-4-chlorobenzamide
4-chloro-N-({5-[(3-{[(1-hydroxycyclohexyl)methyl]amino}pyrrolidin-1-yl)sulfonyl]thien-2-yl}methyl)benzamide
N-{[5-({3-[(1-adamantylmethyl)amino]pyrrolidin-1-yl}sulfonyl)thien-2-yl]methyl}-4-chlorobenzamide
4-chloro-N-{[5-({3-[(3-morpholin-4-ylpropyl)amino]pyrrolidin-1-yl}sulfonyl)thien-2-yl]methyl}benzamide
4-chloro-N-{[5-({3-[(2-pyridin-2-ylethyl)amino]pyrrolidin-1-yl}sulfonyl)thien-2-yl]methyl}benzamide
4-chloro-N-{[5-({3-[(2-piperidin-1-ylethyl)amino]pyrrolidin-1-yl}sulfonyl)thien-2-yl]methyl}benzamide
4-chloro-N-{[5-({3-[(2-ethylhexyl)amino]pyrrolidin-1-yl}sulfonyl)thien-2-yl]methyl}benzamide
4-chloro-N-[(5-{[3-(octylamino)pyrrolidin-1-yl]sulfonyl}thien-2-yl)methyl]benzamide
methyl (2S)-1-[(5-{[(4-chlorobenzoyl)amino]methyl}-2-thienyl)sulfonyl]-4-(hexylamino)-2-pyrrolidinecarboxylate
3-methoxy-N-{[5-({4-[(pentylamino)methyl]piperidin-1-yl}sulfonyl)thien-2-yl]methyl}benzamide
N-{[5-({4-[2-(butylamino)ethyl]piperidin-1-yl}sulfonyl)thien-2-yl]methyl}-3-methoxybenzamide
N-{[5-({4-[(4-butylanilino)methyl]-1-piperidinyl}sulfonyl)-2-thienyl]methyl}-3-methoxybenzamide
4-chloro-N-{[5-({4-[hexyl(methyl)amino]piperidin-1-yl}sulfonyl)thien-2-yl]methyl}benzamide
4-chloro-N-{[5-({4-[(cyclohexylmethyl)(hexyl)amino]piperidin-1-yl}sulfonyl)thien-2-yl]methyl}benzamide
N-{[5-({4-[benzyl(hexyl)amino]piperidin-1-yl}sulfonyl)thien-2-yl]methyl}-4-chlorobenzamide
4-chloro-N-{[5-({4-[hexyl(pyridin-3-ylmethyl)amino]piperidin-1-yl}sulfonyl)thien-2-yl]methyl}benzamide
4-chloro-N-{[5-({4-[hexyl(pyridin-4-ylmethyl)amino]piperidin-1-yl}sulfonyl)thien-2-yl]methyl}benzamide
4-chloro-N-{[5-({4-[hexyl(pyridin-2-ylmethyl)amino]piperidin-1-yl}sulfonyl)thien-2-yl]methyl}benzamide
N-{[5-({4-[butyl(hexyl)amino]piperidin-1-yl}sulfonyl)thien-2-yl]methyl}-4-chlorobenzamide
4-chloro-N-{[5-({4-[hexyl(3-phenylpropyl)amino]piperidin-1-yl}sulfonyl)thien-2-yl]methyl}benzamide
4-chloro-N-{[5-({4-[hexyl(2-phenylethyl)amino]piperidin-1-yl}sulfonyl)thien-2-yl]methyl}benzamide
N-{[5-({4-[[(5-bromo-2-furyl)methyl](hexyl)amino]piperidin-1-yl}sulfonyl)thien-2-yl]methyl}-4-chlorobenzamide
3-methoxy-N-({5-[(4-{methyl[4-(trifluoromethyl)benzyl]amino}-1-piperidinyl)sulfonyl]-2-thienyl}methyl)benzamide
4-chloro-N-{[5-({4-[(3-chlorobenzyl)amino]piperidin-1-yl}sulfonyl)thien-2-yl]methyl}benzamide
3-methoxy-N-({5-[(4-{[4-(trifluoromethyl)benzyl]amino}piperidin-1-yl)sulfonyl]thien-2-yl}methyl)benzamide
3-methoxy-N-{[5-({4-[(3-methylbenzyl)amino]piperidin-1-yl}sulfonyl)thien-2-yl]methyl}benzamide
3-methoxy-N-{[5-({4-[(4-propylbenzyl)amino]piperidin-1-yl}sulfonyl)thien-2-yl]methyl}benzamide
3-methoxy-N-({5-[(4-{[3-(trifluoromethyl)benzyl]amino}piperidin-1-yl)sulfonyl]thien-2-yl}methyl)benzamide
3-methoxy-N-({5-[(4-{[4-(trifluoromethoxy)benzyl]amino}piperidin-1-yl)sulfonyl]thien-2-yl}methyl)benzamide
N-({5-[(4-{[4-(difluoromethoxy)benzyl]amino}piperidin-1-yl)sulfonyl]thien-2-yl}methyl)-3-methoxybenzamide
3-methoxy-N-{[5-({4-[(2,3,4,5,6-pentamethylbenzyl)amino]piperidin-1-yl}sulfonyl)thien-2-yl]methyl}benzamide
3-methoxy-N-{[5-({4-[(4-propoxybenzyl)amino]piperidin-1-yl}sulfonyl)thien-2-yl]methyl}benzamide
N-{[5-({4-[(4-butoxybenzyl)amino]piperidin-1-yl}sulfonyl)thien-2-yl]methyl}-3-methoxybenzamide
3-methoxy-N-{[5-({4-[(4-methoxybenzyl)amino]piperidin-1-yl}sulfonyl)thien-2-yl]methyl}benzamide
3-methoxy-N-{[5-({4-[(pyridin-4-ylmethyl)amino]piperidin-1-yl}sulfonyl)thien-2-yl]methyl}benzamide
3-methoxy-N-{[5-({4-[(pyridin-2-ylmethyl)amino]piperidin-1-yl}sulfonyl)thien-2-yl]methyl}benzamide
3-methoxy-N-{[5-({4-[(pyridin-3-ylmethyl)amino]piperidin-1-yl}sulfonyl)thien-2-yl]methyl}benzamide
N-{[5-({4-[(4-tert-butylbenzyl)amino]piperidin-1-yl}sulfonyl)thien-2-yl]methyl}-3-methoxybenzamide
N-{[5-({4-[(3-ethoxybenzyl)amino]piperidin-1-yl}sulfonyl)thien-2-yl]methyl}-3-methoxybenzamide
3-methoxy-N-{[5-({4-[(4-phenoxybenzyl)amino]piperidin-1-yl}sulfonyl)thien-2-yl]methyl}benzamide
3-methoxy-N-[(5-{[4-({4-[(trifluoromethyl)sulfanyl]benzyl}amino)piperidin-1-yl]sulfonyl}thien-2-yl)methyl]benzamide
3-methoxy-N-({5-[(4-{[4-(methylsulfonyl)benzyl]amino}-1-piperidinyl)sulfonyl]-2-thienyl}methyl)benzamide
N-({5-[(4-{[3,5-bis(trifluoromethyl)benzyl]amino}-1-piperidinyl)sulfonyl]-2-thienyl}methyl)-3-methoxybenzamide
N-({5-[(4-{[2,5-bis(trifluoromethyl)benzyl]amino}-1-piperidinyl)sulfonyl]-2-thienyl}methyl)-3-methoxybenzamide
N-({5-[(4-{[4-(ethylsulfanyl)benzyl]amino}-1-piperidinyl)sulfonyl]-2-thienyl}methyl)-3-methoxybenzamide
3-methoxy-N-[(5-{[4-({3-[(trifluoromethyl)sulfanyl]benzyl}amino)-1-piperidinyl]sulfonyl}-2-thienyl)methyl]benzamide
N-({5-[(4-{[(2,2-difluoro-1,3-benzodioxol-5-yl)methyl]amino}-1-piperidinyl)sulfonyl]-2-thienyl}methyl)-3-methoxybenzamide
N-{[5-({4-[(4-iodobenzyl)amino]-1-piperidinyl}sulfonyl)-2-thienyl]methyl}-3-methoxybenzamide
N-({5-[(4-{[4-(benzyloxy)benzyl]amino}-1-piperidinyl)sulfonyl]-2-thienyl}methyl)-3-methoxybenzamide
N-{[5-({4-[(mesitylmethyl)amino]-1-piperidinyl}sulfonyl)-2-thienyl]methyl}-3-methoxybenzamide
N-{[5-({4-[(4-chlorobenzyl)amino]-1-piperidinyl}sulfonyl)-2-thienyl]methyl}-3-methoxybenzamide
N-{[5-({4-[(4-ethylbenzyl)amino]-1-piperidinyl}sulfonyl)-2-thienyl]methyl}-3-methoxybenzamide
3-methoxy-N-{[5-({4-[(4-pentylbenzyl)amino]-1-piperidinyl}sulfonyl)-2-thienyl]methyl}benzamide
3-methoxy-N-[(5-{[4-({1-[4-(trifluoromethyl)phenyl]ethyl}amino)-1-piperidinyl]sulfonyl}-2-thienyl)methyl]benzamide
3-methoxy-N-{[5-({4-[(4-methylbenzyl)amino]-1-piperidinyl}sulfonyl)-2-thienyl]methyl}benzamide
N-{[5-({4-[(4-butylbenzyl)amino]-1-piperidinyl}sulfonyl)-2-thienyl]methyl}-3-methoxybenzamide
N-{[5-({4-[(4-isopropylbenzyl)amino]-1-piperidinyl}sulfonyl)-2-thienyl]methyl}-3-methoxybenzamide
N-{[5-({4-[(4-isobutylbenzyl)amino]-1-piperidinyl}sulfonyl)-2-thienyl]methyl}-3-methoxybenzamide
N-({5-[(4-{[(1-hydroxy-1lambda˜5˜-pyridin-4-yl)methyl]amino}-1-piperidinyl)sulfonyl]-2-thienyl}methyl)-3-methoxybenzamide
N-{[5-({4-[(2,3-dihydro-1,4-benzodioxin-6-ylmethyl)amino]-1-piperidinyl}sulfonyl)-2-thienyl]methyl}-3-methoxybenzamide
N-{[5-({4-[(2,3-dihydro-1-benzofuran-5-ylmethyl)amino]-1-piperidinyl}sulfonyl)-2-thienyl]methyl}-3-methoxybenzamide
4-chloro-N-{[5-({4-[(4-propylbenzyl)amino]-1-piperidinyl}sulfonyl)-2-thienyl]methyl}benzamide
4-chloro-N-({5-[(4-{[4-(trifluoromethoxy)benzyl]amino}-1-piperidinyl)sulfonyl]-2-thienyl}methyl)benzamide
4-chloro-N-({5-[(4-{[4-(difluoromethoxy)benzyl]amino}-1-piperidinyl)sulfonyl]-2-thienyl}methyl)benzamide
4-chloro-N-{[5-({4-[(4-propoxybenzyl)amino]-1-piperidinyl}sulfonyl)-2-thienyl]methyl}benzamide
N-{[5-({4-[(4-butoxybenzyl)amino]-1-piperidinyl}sulfonyl)-2-thienyl]methyl}-4-chlorobenzamide
4-chloro-N-{[5-({4-[(4-quinolinylmethyl)amino]-1-piperidinyl}sulfonyl)-2-thienyl]methyl}benzamide
N-{[5-({4-[(4-tert-butylbenzyl)amino]-1-piperidinyl}sulfonyl)-2-thienyl]methyl}-4-chlorobenzamide
4-chloro-N-{[5-({4-[(4-phenoxybenzyl)amino]-1-piperidinyl}sulfonyl)-2-thienyl]methyl}benzamide
4-chloro-N-[(5-{[4-({4-[(trifluoromethyl)sulfanyl]benzyl}amino)-1-piperidinyl]sulfonyl}-2-thienyl)methyl]benzamide
4-chloro-N-({5-[(4-{[4-(trifluoromethyl)benzyl]amino}-1-piperidinyl)sulfonyl]-2-thienyl}methyl)benzamide
3-methoxy-N-({5-[(4-{[2-(trifluoromethyl)benzyl]amino}-1-piperidinyl)sulfonyl]-2-thienyl}methyl)benzamide
3-methoxy-N-[(5-{[4-({[6-(trifluoromethyl)-3-pyridinyl]methyl}amino)-1-piperidinyl]sulfonyl}-2-thienyl)methyl]benzamide
N-[(5-{[4-(benzylamino)-1-piperidinyl]sulfonyl}-2-thienyl)methyl]-3-methoxybenzamide
3-methoxy-N-[(5-{[4-({1-[4-(trifluoromethyl)phenyl]propyl}amino)-1-piperidinyl]sulfonyl}-2-thienyl)methyl]benzamide
3-methoxy-N-[(5-{[4-({1-methyl-1-[4-(trifluoromethyl)phenyl]ethyl}amino)-1-piperidinyl]sulfonyl}-2-thienyl)methyl]benzamide
4-chloro-N-[(5-{[4-({1-[4-(trifluoromethyl)phenyl]ethyl}amino)-1-piperidinyl]sulfonyl}-2-thienyl)methyl]benzamide
4-chloro-N-[(5-{[4-({1-methyl-1-[4-(trifluoromethyl)phenyl]ethyl}amino)-1-piperidinyl]sulfonyl}-2-thienyl)methyl]benzamide
4-chloro-N-[(5-{[2-({[4-(trifluoromethyl)benzyl]amino}methyl)-1-pyrrolidinyl]sulfonyl}-2-thienyl)methyl]benzamide
4-chloro-N-[(5-{[(3R)-3-({[4-(trifluoromethyl)benzyl]amino}methyl)pyrrolidinyl]sulfonyl}-2-thienyl)methyl]benzamide
4-chloro-N-({5-[(3-{[4-(trifluoromethyl)benzyl]amino}-1-piperidinyl)sulfonyl]-2-thienyl}methyl)benzamide
4-chloro-N-{[5-({3-[(hexylamino)methyl]-1-piperidinyl}sulfonyl)-2-thienyl]methyl}benzamide
4-chloro-N-({5-[(3-{[4-(trifluoromethyl)benzyl]amino}-1-pyrrolidinyl)sulfonyl]-2-thienyl}methyl)benzamide
4-chloro-N-{[5-({(3R)-3-[(hexylamino)methyl]pyrrolidinyl}sulfonyl)-2-thienyl]methyl}benzamide
4-chloro-N-[(5-{[3-({[4-(trifluoromethyl)benzyl]amino}methyl)-1-piperidinyl]sulfonyl}-2-thienyl)methyl]benzamide
2-oxo-N-({5-[(4-{[4-(trifluoromethyl)benzyl]amino}-1-piperidinyl)sulfonyl]-2-thienyl}methyl)-1,2-dihydro-3-pyridinecarboxamide
N-[(5-{[4-(hexylamino)-1-piperidinyl]sulfonyl}-2-thienyl)methyl]-2-oxo-1,2-dihydro-3-pyridinecarboxamide
N-[(5-{[4-(hexylamino)-1-piperidinyl]sulfonyl}-2-thienyl)methyl]-2-hydroxybenzamide
2-hydroxy-N-({5-[(4-{[4-(trifluoromethyl)benzyl]amino}-1-piperidinyl)sulfonyl]-2-thienyl}methyl)benzamide
N-[(5-{[4-(hexylamino)-1-piperidinyl]sulfonyl}-2-thienyl)methyl]-2-thioxo-1,2-dihydro-3-pyridinecarboxamide
2-thioxo-N-({5-[(4-{[4-(trifluoromethyl)benzyl]amino}-1-piperidinyl)sulfonyl]-2-thienyl}methyl)-1,2-dihydro-3-pyridinecarboxamide
N-[(5-{[4-(butylamino)-1-piperidinyl]sulfonyl}-2-thienyl)methyl]-2-oxo-1,2-dihydro-3-pyridinecarboxamide
N-({5-[(4-{ethyl[4-(trifluoromethyl)benzyl]amino}-1-piperidinyl)sulfonyl]-2-thienyl}methyl)-3-methoxybenzamide
4-chloro-N-[(5-{[4-({imino[4-(trifluoromethyl)phenyl]methyl}amino)-1-piperidinyl]sulfonyl}-2-thienyl)methyl]benzamide
1-[(5-{[(4-chlorobenzoyl)amino]methyl}-2-thienyl)sulfonyl]-4-(hexylamino)proline ethyl 2-{[4-(hexylamino)piperidin-1-yl]sulfonyl}-5-{[(3-methoxybenzoyl)amino]methyl}thiophene-3-carboxylate
N-{[5-{[4-(hexylamino)piperidin-1-yl]sulfonyl}-4-(trimethylsilyl)thien-2-yl]methyl}-3-methoxybenzamide
N-({5-{[4-(hexylamino)piperidin-1-yl]sulfonyl}-4-[hydroxy(phenyl)methyl]thien-2-yl}methyl)-3-methoxybenzamide
5-[(3-Methoxy-benzoylamino)-methyl]-2-[4-(4-trifluoromethyl-benzylamino)-piperidine-1-sulfonyl]-thiophene-3-carboxylic acid ethyl ester
N-[(4-chloro-5-{[4-(hexylamino)piperidin-1-yl]sulfonyl}thien-2-yl)methyl]-3-methoxybenzamide.

The compounds of formula (III) may be obtained according to the methods described in any of WO 01/23378, WO 02/28856 and WO 02/26733.

In a further embodiment the JNK inhibitors may be a pyrazoloanthrone derivative as shown in formula (C) (WO 01/12609):

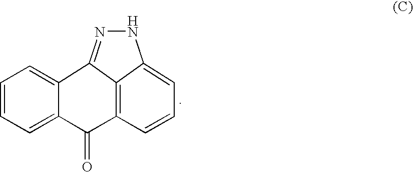

In a further embodiment the JNK inhibitors may have the formula (IV) (WO 03/018022):

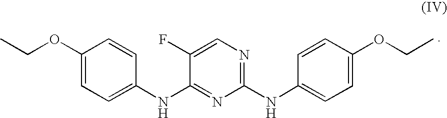

The invention further relates to pharmaceutical compositions, particularly useful for prevention and/or treatment of skin diseases, preferably skin inflammatory skin diseases and more preferably psoriasis, which comprise a therapeutically effective amount of JNK inhibitor and a pharmaceutical excipient.

The invention also relates to a method of treatment comprising administering to a patient in need thereof a therapeutically effective amount of a JNK inhibitor.

The pharmaceutical compositions of the present invention can be administered by a variety of routes including oral, rectal, transdermal, subcutaneous, intravenous, intramuscular and intranasal. The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the JNK inhibitor is usually a minor component (from about 0.1 to about 50% by weight or preferably from about 1 to about 40% by weight) with the remainder being various vehicles or carriers and processing aids helpful for forming the desired dosing form.

The route of administration, which is preferred according to the invention is administration by oral route.

Liquid forms suitable for oral administration may include a suitable aqueous or nonaqueous vehicle with buffers, suspending and dispensing agents, colorants, flavors and the like.

Solid forms may include, for example, any of the following ingredients, or compounds of a similar nature: a binder such as microcrystalline cellulose, gum tragacanth or gelatine; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as pepper-mint, methyl salicylate, or orange flavoring.

Injectable compositions are typically based upon injectable sterile saline or phosphate-buffered saline or other injectable carriers known in the art. As above-mentioned, the JNK inhibitor in such compositions is typically a minor component, frequently ranging between 0.05 to 10% by weight with the remainder being the injectable carrier and the like.

The above-described components for orally administered or injectable compositions are merely representative. Further materials as well as processing techniques and the like are set out in Part 5 of Remington's Pharmaceutical Sciences, 20^{th} Edition, 2000, Marck Publishing Company, Easton, Pa., which is incorporated herein by reference.

The compounds of this invention can also be administered in sustained release forms or from sustained release drug delivery systems. A description of representative sustained release materials can also be found in the incorporated materials in Remington's Pharmaceutical Sciences.

The definition of “pharmaceutically acceptable” is meant to encompass any carrier, which does not interfere with effectiveness of the biological activity of the active ingredient and that is not toxic to the host to which it is administered. For example, for parenteral administration, JNK inhibitor may be formulated in a unit dosage form for injection in vehicles such as saline, dextrose solution, serum albumin and Ringer's solution.

For parenteral (e.g. intravenous, subcutaneous, intramuscular) administration, JNK inhibitors can be formulated as a solution, suspension, emulsion or lyophilized powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The therapeutically effective amounts of a JNK inhibitor will be a function of many variables, including the type of inhibitor, the affinity of the inhibitor for JNK, any residual cytotoxic activity exhibited by the JNK inhibitor, the route of administration or the clinical condition of the patient.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Depending upon the particular condition, or disease, to be treated or prevented, additional therapeutic agents, which are normally administered to treat or prevent that condition, may also be present in the compositions of this invention. As used herein, additional therapeutic agents that are normally administered to treat or prevent a particular disease, or condition, are known as “appropriate for the disease, or condition, being treated”.

The dose of the JNK Inhibitor is adjusted between 10 and 100 mg/kg, preferably to 40-80 mg/kg of body weight.

In a preferred embodiment of the present invention, the JNK inhibitor is used in about 0.1 to 100 mg/kg of body weight, or about 1 to 50 mg/kg of body weight or about 5 to 10 mg/kg of body weight.

According to the invention, a JNK inhibitor can be administered prophylactically or therapeutically to an individual prior to, simultaneously or sequentially with other therapeutic regimens or agents (e.g. multiple drug regimens), in a therapeutically effective amount. Active agents that are administered simultaneously with other therapeutic agents can be administered in the same or different compositions.

Therefore, another aspect of the invention further related to a the use of a therapeutically effective amount a JNK inhibitor alone or in combination with an additional therapeutic agent selected from Methotrexate, efalizumab, Cyclosporine, Corticosteroids, Topical retinoids for the treatment of a skin disease.

A further aspect of the invention further relates to a method for treating a skin disease comprising administering to a patient in need thereof an effective amount of a JNK inhibitor optionally together with a pharmaceutically acceptable carrier.

The present invention will now be illustrated by the example, which is not intended to be limiting in any way.

### EXAMPLES

### Example 1

### Contact Hypersensitivity Model in Mice (CHS)

### Introduction

Contact hypersensitivity (CHS) is a T-cell mediated model of skin inflammation related disorders. The immune response is mediated by IFNγ-producing Th1/Tc1 cells and is a model of psoriasis and allergic dermatitis. In this model the hapten DNFB (dinitrofluorobenzene), which induces a type-1 cytokine response was used.

Psoriatic skin shows markedly increased keratinocyte proliferation and altered differentiation with various abnormal signaling pathways. Immunohistochemical and Western blot analyses studies showed increased mitogen activated kinases (MAPKs) expression in the nuclei of psoriatic patients epidermis. Since alterations in specific signal transduction pathways may explain the hyperproliferation and abnormal keratinocytes differentiation as well as the increased expression of inflammatory cytokines seen in skin diseases, was investigated the Contact hypersensitivity model (CHS) signaling and cytokine release during a time course.

### Materials and Method

Balb/C female mice of about 8-12 weeks of age (18-22 g b.w.) were used for the Contact Hypersensitivity model. They were from Charles River Italia (Calco, Italy) and housed under the following constant environmental conditions: temperature 22° C.±2, relative humidity 55%±10, 15-20 air changes per hour (filtered on HEPA 99.99%) and artificial light with a 12-hour circadian cycle (7 a.m.-7 p.m.). All in vivo studies were performed according to the European Council Directive 86/609/EEC and the Italian Ministry guidelines for the care and use of experimental animals (decree #116/92). All the experimental protocols were authorized by the Italian Ministry of Health.

### Chemicals and Reagents

All chemicals were purchased from Sigma-Aldrich (St. Louis, Mo., USA) unless otherwise specified. Antibodies used Western Blotting were obtained from Cell Signaling Technology (Beverly, Mass., USA). The JNK inhibitor 1,3-benzothiazol-2-yl(2-{[4-(morpholin-4-ylmethyl)benzyl]oxy}pyrimidin-4-yl)acetonitrile was from Serono Pharmaceutical Research Institute, Geneva, Switzerland. For the in vivo studies, the JNK inhibitor and dexamethasone were suspended in deionised water as vehicle.

### Contact Hypersensitization Model (CHS)

For the induction of CHS, 8-12 weeks old Balb/C mice, were sensitized by applying once a sensitizing solution of 35 μl of 0.5% DNFB in acetone/olive oil (4:1) on the shaved back. 6 mice were used as negative control (unsensitized animals). After 5 days, sensitized and unsensitized control animals were challenged (CH) by applying 15 μl of 0.2% DNFB in acetone/olive oil (4:1) to the dorsal and ventral side of the right ear. As control, the left ear was challenged with an equal volume of vehicle (acetone/olive oil). Ear thickness was monitored using a caliper at day 0, at day 5 (4 h, 8 h), at day 6 (24 h) and at day 7 (48 h). A non-sensitized but challenged control group (n=6) was included. Ear thickness of both ears was measured at three point each, ear swelling was evaluated according to the following formula:

[(Tₙ−T₅)right ear]−[(Tₙ−T₅)left ear]

where Tₙ and T₅ represent values of averaged ear thickness at day n of investigation and at day 5 pre challenge, respectively.
At every time point, 6 mice were sacrificed and challenged ear homogenated for cytokine detection and Western Blot analysis.

### JNK Inhibitor in the Contact Hypersensitization Model (CHS)

Four groups (n=6) of mice were sensitized and 30 minutes before challenge treated respectively with vehicle (water for injection), JNK Inhibitor at 10 and 30 mg/kg p.o. and dexamethasone at 1 mg/kg s.c. as reference compound. A non-sensitized but challenged control group (n=6) was included. After 24 h mice were sacrificed and challenged ear snap frozen in liquid nitrogen and homogenated for cytokine detection and Western Blot analysis.

### Cytokine Detection

Challenged ears were snap frozen, homogenated and resuspended with ice-cold lysis buffer (PBS 1×, 2% w/v SDS, 50 mM DTT). After sonication, lysates were centrifuged, protein concentration determined and 100 μg used for cytokine analysis with BD mouse CBA (Cytometric Bead Array, BD) Inflammation and Th1/Th2 kits and processed as described by the technical brochure. Samples were analysed using a FACS Calibur and Cell Quest software (BD Biosciences, San Jose, Calif., USA).

### Western Blotting

Challenged ears were snap frozen, homogenated and resuspended with ice-cold lysis buffer (PBS 1×, 2% w/v SDS, 50 mM DTT). After sonication, lysates were centrifuged, protein concentration determined and 100 μg of proteins separated by electrophoresis on 10% SDS-PAGE and transferred onto a polyvinylidene difluoride-plus membrane. After blocking with 5% milk, the immunoblots were probed with a 1:1000 dilution anti phosphorylated- and total-AKT, ERK1/2, c-jun, p38MAPK, caspase-3 and cleaved caspase-3 antibody (Cell Signaling Technology) overnight at 4° C., followed by a 1 hour incubation at room temperature with the corresponding secondary antibodies. The blots were visualized with ECL-plus reagent. Phospho-immunoblots were then stripped with strip buffer at 50° C. for 30 minutes and reblotted for total protein (Cell Signaling Technology). The volume of the protein bands was quantified by a Bio-Rad ChemiDoc™ EQ densitometer and a Bio-Rad Quantity One® software (Bio-Rad Laboratories, Hercules, Calif., USA).

### Statistical Analysis

All values in the text and figures are presented as mean±SEM of (n) independent experiments. All data were analysed by one-way ANOVA followed by Tukey test. P values <0.05 were considered statistically significant. S-Plus 2000 v. 4.6 statistical software was used (Mathsoft Inc., Seattle, Wash., USA).

### Results

### Contact Hypersensitivity Model: Ear Swelling and Cytokine Detection Time Course

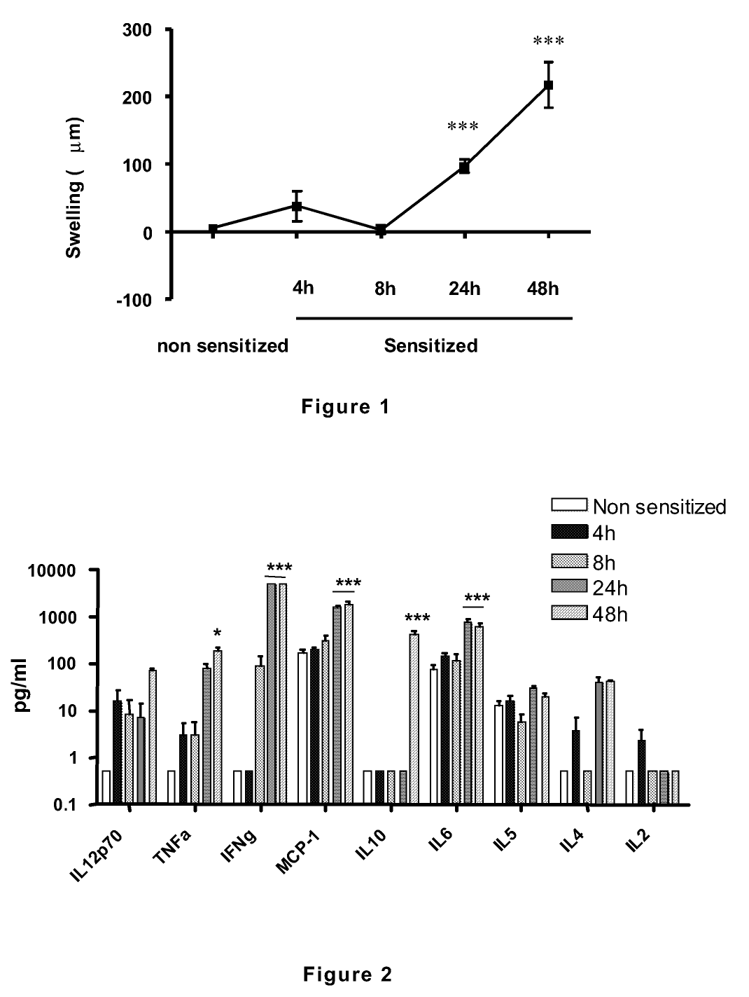

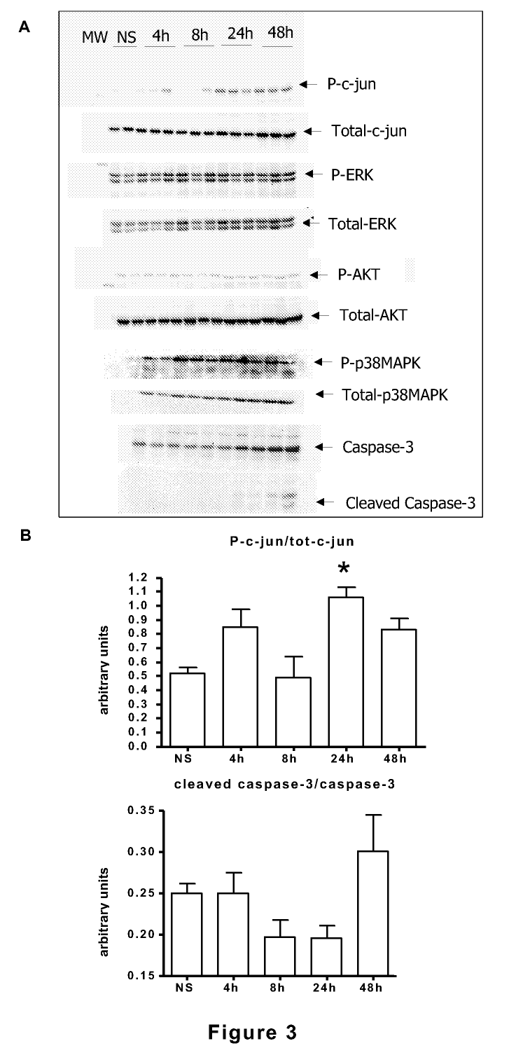

Ear swelling and inflammatory cytokines presented the same trend of increase during the CHS time course. Ear thickness, IFNγ, MCP-1 and IL6 dramatically rose up (p<0.001) at 24 h and 48 h, TNFα and IL10 increased at 48 h (respectively p<0.05 and p<0.001, one-way ANOVA followed by Tukey test) (FIG. 1-2).

No variation in phosphorylation- and total-AKT, ERK and p38 MAPK along the whole time-course. On the contrary was observed that c-jun-phosphorylation status reached a peak 24 h after challenge (p<0.05) and caspase-3 cleavage was activated at 48 h after challenge. No differences in total-c-jun and caspase-3 were shown. (FIG. 3). These data suggested the pivotal role of c-jun-N terminal Kinase (JNK) in the inflammatory processes of the CHS model.

### JNK Inhibitor in the Contact Hypersensitivity Model

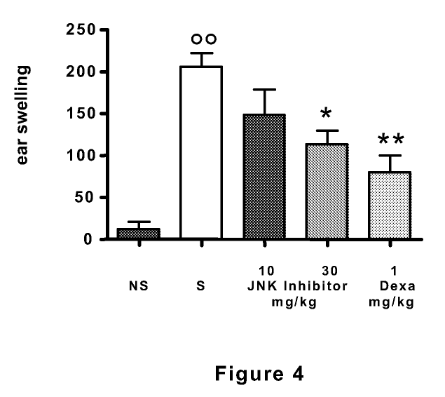

In order to investigate the role of JNK in the CHS model, we administered 1,3-benzothiazol-2-yl(2-{[4-(morpholin-4-ylmethyl)benzyl]oxy}pyrimidin-4-yl)acetonitrile, a non JNK isoform specific inhibitor. Four groups (n=6) of mice were sensitized and 30 minutes before challenge treated respectively with vehicle (water for injection), JNK inhibitor at 10 and 30 mg/kg p.o. and dexamethasone at 1 mg/kg s.c. as reference compound. A non-sensitized but challenged control group (n=6) was included. JNK inhibitor at 30 mg/kg p.o. and dexamethasone significantly reduced ear swelling (p<0.05 and p<0.01 respectively). (FIG. 4)

This modulation can lead to therapeutic efficacy by e.g. release of soluble mediators. Extended analysis of immune responses after JNK inhibition should give insight of JNK involvement and potential therapeutic impact.

### REFERENCE LIST

Davis, R. J. (2000). Signal transduction by the JNK group of MAP kinases. Cell 103, 239-252.
de la Monte, S. M., Ganju, N., Feroz, N., Luong, T., Banerjee, K., Cannon, J., and Wands, J. R. (2000). Oxygen free radical injury is sufficient to cause some Alzheimer-type molecular abnormalities in human CNS neuronal cells. J Alzheimers. Dis. 2, 261-281.
Dumitru, C. D., Ceci, J. D., Tsatsanis, C., Kontoyiannis, D., Stamatakis, K., Lin, J. H., Patriotis, C., Jenkins, N. A., Copeland, N. G., Kollias, G., and Tsichlis, P. N. (2000). TNF-alpha induction by LPS is regulated posttranscriptionally via a Tpl2/ERK-dependent pathway. Cell 103, 1071-1083.
Gupta, S., Barrett, T., Whitmarsh, A. J., Cavanagh, J., Sluss, H. K., Derijard, B., and Davis, R. J. (1996). Selective interaction of JNK protein kinase isoforms with transcription factors. EMBO J. 15, 2760-2770.
Han, Z., Boyle, D. L., Chang, L., Bennett, B., Karin, M., Yang, L., Manning, A. M., and Firestein, G. S. (2001). c-Jun N-terminal kinase is required for metalloproteinase expression and joint destruction in inflammatory arthritis. J Clin Invest 108, 73-81.
Kempiak, S. J., Hiura, T. S., and Nel, A. E. (1999). The Jun kinase cascade is responsible for activating the CD28 response element of the IL-2 promoter: proof of cross-talk with the I kappa B kinase cascade. J Immunol 162, 3176-3187.
Nishina, H., Bachmann, M., Oliveira-dos-Santos, A. J., Kozieradzki, I., Fischer, K. D., Odermatt, B., Wakeham, A., Shahinian, A., Takimoto, H., Bernstein, A., Mak, T. W., Woodgett, J. R., Ohashi, P. S., and Penninger, J. M. (1997). Impaired CD28-mediated interleukin 2 production and proliferation in stress kinase SAPK/ERK1 kinase (SEK1)/mitogen-activated protein kinase kinase 4 (MKK4)-deficient T lymphocytes . J Exp Med 186, 941-953.
Takahashi, H., Ibe, M., Nakamura, S., Ishida-Yamamoto, A., Hashimoto, Y., and Iizuka, H. (2002). Extracellular regulated kinase and c-Jun N-terminal kinase are activated in psoriatic involved epidermis. J. Dermatol. Sci 30, 94-99.
Zhu, X., Raina, A. K., Rottkamp, C. A., Aliev, G., Perry, G., Boux, H., and Smith, M. A. (2001). Activation and redistribution of c-jun N-terminal kinase/stress activated protein kinase in degenerating neurons in Alzheimer's disease . J. Neurochem. 76, 435-441.
EP 160,699
EP 211,894
EP 322,438
WO 00/35906
WO 00/35909
WO 00/3592
WO 00/64872
WO 00/75118
WO 01/12609
WO 01/12621
WO 01/23378
WO 01/23379
WO 01/23382
WO 01/47920, EP 110957
WO 02/079197
WO 02/26733
WO 02/28856
WO 02/46184
WO 03/018022
WO 03/047570
WO 2005/097116
WO 2006/047354
WO 92/13095

1-18. (canceled)

19. A method of treating psoriasis or allergic dermatitis comprising the administration of a therapeutically effective amount of a composition comprising a JNK inhibitor to an individual in need of treatment.

20. The method according to claim 19, wherein the JNK inhibitor is a benzothiazole derivative according to formula (I) 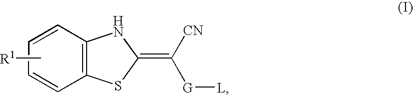
tautomers, geometrical isomers, enantiomers, diastereomers, racemates and pharmaceutically acceptable salts thereof, wherein:
G is a pyrimidinyl group;
L is C₁-C₆-alkoxy, or an amino group, or a 3-8 membered heterocycloalkyl, containing at least one heteroatom selected from N, O, S;
R¹ is selected from the group consisting of hydrogen, sulfonyl, amino, C₁-C₆-alkyl, C₂-C₆-alkenyl, C₂-C₆-alkynyl or C₁-C₆-alkoxy, aryl, halogen, cyano and hydroxy.

21. The method according to claim 20, wherein R¹ is H or C₁-C₃ alkyl.

22. The method according to claim 20, wherein the JNK inhibitor has formulae (Ia), (Ia′) or (Ia″): 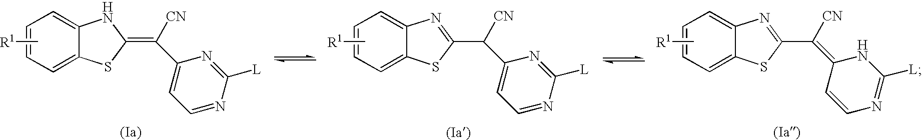
wherein R¹ is selected from the group consisting of hydrogen, sulfonyl, amino, C₁-C₆-alkyl, C₂-C₆-alkenyl, C₂-C₆-alkynyl or C₁-C₆-alkoxy, aryl, halogen, cyano and hydroxy; L is an amino group of the formula —NR³R⁴ wherein R³ and R⁴ are each independently from each other H, C₁-C₆-alkyl, C₂-C₆-alkenyl, C₂-C₆-alkynyl, C₁-C₆-alkoxy, aryl, heteroaryl, saturated or unsaturated 3-8-membered cycloalkyl, 3-8-membered heterocycloalkyl, (wherein said cycloalkyl, heterocycloalkyl, aryl or heteroaryl groups may be fused with 1-2 further cycloalkyl, heterocycloalkyl, aryl or heteroaryl group), C₁-C₆-alkyl aryl, C₁-C₆-alkyl heteroaryl, C₂-C₆-alkenyl aryl, C₂-C₆-alkenyl heteroaryl, C₂-C₆-alkynyl aryl, C₂-C₆-alkynyl heteroaryl, C₁-C₆-alkyl cycloalkyl, C₁-C₆-alkyl heterocycloalkyl, C₂-C₆-alkenyl cycloalkyl, C₂-C₆-alkenyl heterocycloalkyl, C₂-C₆-alkynyl cycloalkyl, C₂-C₆-alkynyl heterocycloalkyl, or R³ and R⁴ may form a ring together with the nitrogen to which they are bound.

23. The method according to claim 22, wherein R³ is hydrogen or a methyl or ethyl or propyl group and R⁴ is selected from the group consisting of (C₁-C₆)-alkyl, C₁-C₆ alkyl-aryl, C₁-C₆-alkyl-heteroaryl, cycloalkyl, heterocycloalkyl, aryl or heteroaryl and 4-8 membered saturated or unsaturated cycloalkyl.

24. The method according to claim 22, wherein R³ and R⁴ form an unsubstituted or substituted piperazine or a unsubstituted or substituted piperidine or a unsubstituted or substituted morpholine or a unsubstituted or substituted pyrrolidine ring together with the nitrogen to which they are bound, whereby said substituent is selected from the group consisting of C₁-C₆-alkyl, C₂-C₆-alkenyl, C₂-C₆-alkynyl, C₁-C₆-alkoxy, aryl, heteroaryl, saturated or unsaturated 3-8-membered cycloalkyl, 3-8-membered heterocycloalkyl, (wherein said cycloalkyl, heterocycloalkyl, aryl or heteroaryl groups may be fused with 1-2 further cycloalkyl, heterocycloalkyl, aryl or heteroaryl group), C₁-C₆-alkyl aryl, C₁-C₆-alkyl heteroaryl, C₂-C₆-alkenyl aryl, C₂-C₆-alkenyl heteroaryl, C₂-C₆-alkynyl aryl, C₂-C₆-alkynylheteroaryl, C₁-C₆-alkyl cycloalkyl, C₁-C₆-alkyl heterocycloalkyl, C₂-C₆-alkenyl cycloalkyl, C₂-C₆-alkenyl heterocycloalkyl, C₂-C₆-alkynyl cycloalkyl, C₂-C₆-alkynyl heterocycloalkyl.

25. The method according to claim 20, wherein L is selected from: 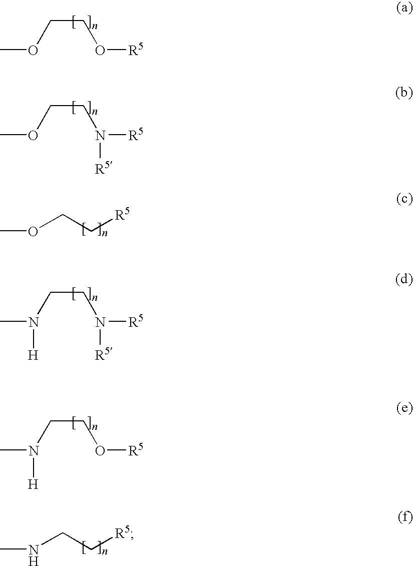
wherein n is 1 to 10, preferably 1 to 6, R⁵ and R^{5′} are independently selected from each other from the group consisting of H, C₁-C₁₀ alkyl, aryl or hetero-aryl, C₁-C₆ alkyl-aryl and C₁-C₆-alkyl-heteroaryl.

26. The method according to claim 22, wherein L is selected from: 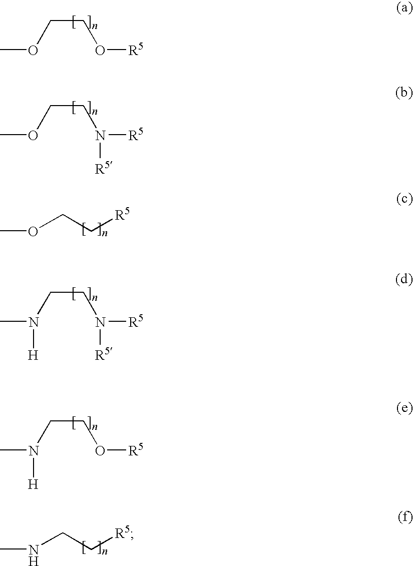
wherein n is 1 to 10, preferably 1 to 6, R⁵ and R^{5′} are independently selected from each other from the group consisting of H, C₁-C₁₀ alkyl, aryl or hetero-aryl, C₁-C₆ alkyl-aryl and C₁-C₆-alkyl-heteroaryl.

27. The method according to claim 20, wherein L is selected from: 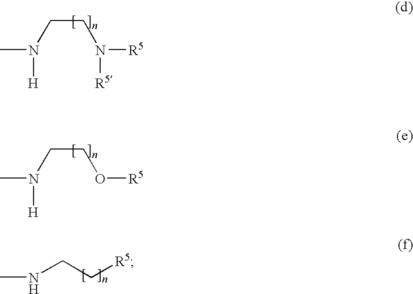
wherein n is 1 to 10, preferably 1 to 6; and R⁵ and R^{5′} are independently selected from each other from the group consisting of H, C₁-C₁₀ alkyl, aryl or hetero-aryl, C₁-C₆ alkyl-aryl and C₁-C₆-alkyl-heteroaryl.

28. The method according to claim 22, wherein L is selected from: 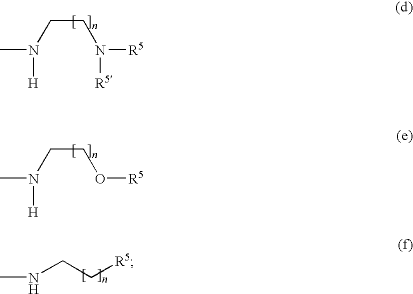
wherein n is 1 to 10, preferably 1 to 6; and R⁵ and R^{5′} are independently selected from each other from the group consisting of H, C₁-C₁₀ alkyl, aryl or hetero-aryl, C₁-C₆ alkyl-aryl and C₁-C₆-alkyl-heteroaryl.

29. The method according to claim 20, wherein the JNK inhibitor is: 1,3-benzothiazol-2-yl(2-{[4-(morpholin-4-ylmethyl)benzyl]oxy}pyrimidin-4-yl)acetonitrile.

30. The method according to claim 20, wherein the JNK inhibitor is a compound of formula (III) 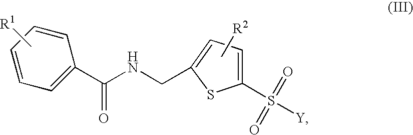
geometrical isomers, enantiomers, diastereomers, racemates and pharmaceutically acceptable salts thereof, wherein Y is a 4-12-membered saturated cyclic or bicyclic alkyl containing at least one nitrogen atom, whereby one nitrogen atom within said ring is forming a bond with the sulfonyl group of formula (III) thus providing the sulfonamide; R¹ is selected from the group consisting of hydrogen, C₁-C₆-alkoxy, C₁-C₆-alkyl, C₂-C₆-alkenyl, C₂-C₆-alkynyl, amino, sulfanyl, sulfinyl, sulfonyl, sulfonyloxy, sulfonamide, acylamino, aminocarbonyl, C₁-C₆ alkoxycarbonyl, aryl, heteroaryl, carboxy, cyano, halogen, hydroxy, nitro and hydrazide; R² is selected from the group consisting of hydrogen, COOR³, —CONR³R^{3′}, OH, a C₁-C₄ alkyl substituted with an OH or amino group, a hydrazido carbonyl group, a sulfate, a sulfonate, an amine and an ammonium salt; with R³, R^{3′} being substituents independently selected from the group consisting of H, C₁-C₆-alkyl, C₂-C₆-alkenyl, aryl, heteroaryl, aryl-C₁-C₆-alkyl, and heteroaryl-C₁-C₆-alkyl.

31. The method according to claim 30, wherein R¹ is selected from the group consisting of hydrogen, halogen, C₁-C₆ alkyl and C₁-C₆ alkoxy.

32. The method according to claim 30, wherein Y is either of the cyclic amines having the general formulae: 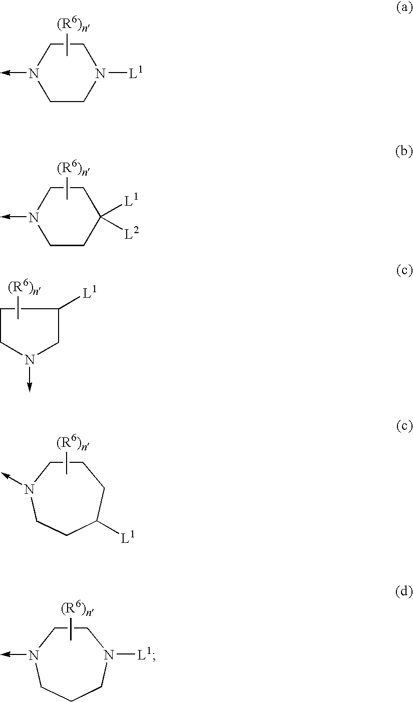
whereby, L¹ and L² are independently selected from each other from the group consisting of C₁-C₆-alkyl, C₂-C₆-alkenyl, C₂-C₆-alkynyl, C₄-C₈-cycloalkyl optionally containing 1-3 heteroatoms and optionally fused with aryl or heteroaryl; or L¹ and L² are independently selected from the group consisting of aryl, heteroaryl, aryl-C₁-C₆-alkyl, heteroaryl-C₁-C₆-alkyl, —C(O)—OR³, —C(O)—R³, —C(O)—NR^{3′}R³, —NR^{3′}R³, —NR^{3′}C(O)R³, —NR^{3′}C(O)NR^{3′}R³, —(SO)R³, —(SO₂)R³, —NSO₂R³, and —SO₂NR^{3′}R³, with R³, R^{3′} being substituents independently selected from the group consisting of H, C₁-C₆-alkyl, C₂-C₆-alkenyl, aryl, heteroaryl, aryl-C₁-C₆-alkyl, and heteroaryl-C₁-C₆-alkyl; or L¹ and L² taken together form a 4-8-membered, saturated cyclic alkyl or heteroalkyl group; and R⁶ is selected from the group consisting of hydrogen, C₁-C₆-alkyl, C₁-C₆-alkoxy, OH, halogen, nitro, cyano, sulfonyl, oxo (═O), and n′ is an integer from 0 to 4, preferably 1 or 2.

33. The method according to claim 32, wherein R⁶ is H, L² is H, L¹ is —NR^{3′}R³; where at least one of R^{3′} and R³ is not hydrogen, but a substituent selected from the group consisting of straight or branched C₄-C₁₈-alkyl, aryl-C₁-C₁₈-alkyl, heteroaryl-C₂-C₁₈-alkyl, C₁-C₁₄-alkyl substituted with a C₃-C₁₂-cycloalkyl or -bicyclo or -tricyloalkyl, and whereby said alkyl chain may contain 1-3 O or S atoms.

34. The method according to claim 32, wherein L¹ is —NHR³; where R³ is a straight or branched C₄-C₁₂-alkyl, preferably a C₆-C₁₂-alkyl, optionally substituted with a cyclohexyl group or a benzyl group.

35. The method according to claim 30, wherein Y is a piperidine group 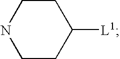
L¹ is —NHR³; where R³ is a straight or branched C₄-C₁₂-alkyl, preferably a C₈-C₁₂-alkyl, or a benzyl group.

36. The method according to claim 20, wherein the JNK inhibitor is used in about 0.1 to 100 mg/kg of body weight, or about 1 to 50 mg/kg of body weight or about 5 to 10 mg/kg of body weight.

37. The method according to claim 20, wherein the administration of JNK inhibitor is via an oral route.

38. The method according to claim 20, wherein said composition further comprises an additional therapeutic agent selected from methotrexate, efalizumab, cyclosporine, corticosteroids, or topical retinoids.
